(19) United States
(12) Patent Application Publication
(10) Pub. No.: US 2025/0313646 A1
(43) Pub. Date: Oct. 9, 2025

(54) GLYCOENGINEERED ANTIBODIES

(71) Applicant: AUGMENT BIOLOGICS, INC., Beaverton, OR (US)

(72) Inventor: Benjamin KELLMAN, Cambridge, MA (US)

(21) Appl. No.: 18/857,720

(22) PCT Filed: Apr. 18, 2023
(86) PCT No.: PCT/US2023/065914

§ 371 Date: Oct. 17, 2024

Related U.S. Application Data
(60) Provisional application No. 63/332,641, filed on Apr. 19, 2022.

(51) Int. Cl. C07K 16/28; C07K 16/32

(52) U.S. Cl. CPC C07K 16/2887; C07K 16/32; C07K 2317/24; C07K 2317/41; C07K 2317/52; C07K 2317/732; C07K 2317/734; C07K 2317/92

(57) ABSTRACT

Disclosed herein are engineered antibodies.

### CROSS-REFERENCE TO RELATED APPLICATIONS

This application claims the benefit of U.S. Provisional Application No. 63/332,641 filed Apr. 19, 2022, the entirety of which is incorporated herein by reference in its entirety.

### SEQUENCE LISTING

The instant application contains a Sequence Listing which has been submitted electronically and is hereby incorporated by reference in its entirety. Said copy, created on Apr. 17, 2023, is named Augment-61849-703601.xml and is 3,066 bytes in size.

### SUMMARY OF THE INVENTION

In one aspect, provided herein are fragment crystallizable (Fc) regions and antibodies comprising Fc regions having altered effector function as compared to human IgG1. For instance, the Fc regions and antibodies have increased or reduced antibody-dependent cell-mediated cytotoxicity (ADCC) function as compared to human IgG1 and/or increased or reduced complement-dependent cytotoxicity (CDC) as compared to human IgG1. In some embodiments, the human IgG1 comprises SEQ ID NO: 1 (ASTKGPSVFPLAPSSKSTSGGTAALGCLVKDYFPEPVTVSWNSGALTSGVHTFPAVLQS SGLYSLSSVVTVPSSSLGTQTYICNVNHKPSNTKVDKKVEPKSCDKTHTCPPCPAPELLG GPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLP PSRDELTKNQVSLTCLVKGFYPSDIAVEWESNGQPENNYKTTPPVLDSDGSFFLYSKLT VDKSRWQQGNVFSCSVMHEALHNHYTQKSLSLSPGK). In some embodiments, the ADCC function of the Fc region comprising increased ADCC is increased at least about 2-fold (e.g., about 2-fold to 100-fold) as compared to human IgG1. In some embodiments, the CDC function of the Fc region comprising increased CDC is increased at least about 2-fold (e.g., about 2-fold to 100-fold) as compared to human IgG1. In some embodiments, the increased effector function is the result of a reduction in fucosylation in the Fc region. In some embodiments, the change in fucosylation in the Fc region occurs due to one or more substitutions in the Fc region as compared to human IgG. In some embodiments herein, fucosylation is core-fucosylation.

In one aspect, are fragment crystallizable (Fc) regions and antibodies comprising Fc regions having altered antibody-dependent cellular phagocytosis (ADCP) function as compared to human IgG1. For instance, the Fc regions and antibodies have increased or reduced ADCP function as compared to human IgG1. In some embodiments, the human IgG1 comprises SEQ ID NO: 1. In some embodiments, the change in ADCP function is the result of a change in Fc glycosylation. In some embodiments, the change in Fc glycosylation occurs due to one or more substitutions in the Fc region as compared to human IgG.

In one aspect, are fragment crystallizable (Fc) regions and antibodies comprising Fc regions having altered antibody-dependent immune response or function as compared to human IgG1. For instance, the Fc regions and antibodies have increased or reduced antibody-dependent immune response or function as compared to human IgG1. In some embodiments, the human IgG1 comprises SEQ ID NO: 1. In some embodiments, the change in antibody-dependent immune response or function is the result of a change in Fc glycosylation. In some embodiments, the change in Fc glycosylation occurs due to one or more substitutions in the Fc region as compared to human IgG.

In one aspect, provided herein are antibody Fc regions comprising a substitution(s): P291E, P291K, P291Q, R292E, R292K, R292Q, Q295E, Y296E, Y296K, Y296Q, R301E, R301K, R301Q, P291I&V303F, V302F&V303F, P291Q&V303F, P291L&V303F, P291L&S304N, V303F&S304N, V303F&S304T, V303F&S304F, P291L&V303W, P291Q&S304N, P291I&V303W, V302W&V303F, V302W&V303W, V302F&V303W, P291I&V302F, P291Q&S304F, P291Q&R292Q, P291L&S304F, P291Q&S304T, P291L&V303Q, V302H&V303F, R292Q&V303F, P291Q&V302F, P291L&V302F, V302Q&V303F, P291I&V303Q, V302Y&V303W, P291I&S304N, V302F&S304N, V302Q&V303W, K290L&P291L, P291Q&T207N, K290L&V303Q, K290L&V303W, K290I&P291L, R292Q&T207N, P291E&T207N, K290L&V303F, K290L&V303Y, K290L&V303I, K290I&V303F, K290I&V303Q, P291L&T207N, K290L&P291Q, K290E&V303F, K290E&R292Q, K290L&V302Q, K290L&R292Q, K290L&V303H, K290L&V302W, K290E&V303Q, K290E&V303Y, K290L&V302I, K290L&V302Y, K290E&V303H, K290E&V302Q, K290L&V302F, K290I&V302Q, K290I&V303W, K290E&P291L, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution decreases core-fucosylation at amino acid N297 as compared to the antibody Fc region without the substitution.

In one aspect, provided herein are antibody Fc regions comprising a substitution at position(s): Q295K, Q295R, Y296F, and/or Y300F, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution increases core-fucosylation at amino acid N297 as compared to the antibody Fc region without the substitution.

In one aspect, provided herein are antibody Fc regions comprising a substitution at position(s): P291R, Y296R, and/or Y296W, according to the EU numbering system; optionally wherein: the substitution does not alter an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution does not alter core-fucosylation at amino acid N297 as compared to the antibody Fc region without the substitution.

In one aspect, provided herein are antibody Fc regions comprising a substitution(s): P291I, P291L, P291V, Y296I, Y296V, S298F, S298H, S298N, S298T, S298W, S298Y, Y300F, Y300H, Y300I, Y300I, Y300L, Y300V, Y300V, Y300W, Y300W, R301H, R301W, V302F, V302H, V302I, V302L, V302Q, V302W, V302Y, V303F, V303H, V303I, V303L, V303Q, V303W, V303Y, S304F, S304H, S304N, S304T, S304W, S304Y, V305H, V305K, V305Q, V305R, or V305W, or any combination thereof, wherein the numbering is according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution alters core-fucosylation at amino acid N297 as compared to the antibody Fc region without the substitution.

In one aspect, provided herein are antibody Fc regions comprising a substitution(s): P291E, P291K, P291Q, R292E, R292K, R292Q, Q295E, Y296E, Y296K, Y296Q, R301E, R301K, R301Q, P291I &V303F, V302F&V303F, P291Q&V303F, P291L&V303F, P291L&S304N, V303F&S304N, V303F&S304T, V303F&S304F, P291L&V303W, P291Q&S304N, P291I&V303W, V302W&V303F, V302W&V303W, V302F&V303W, P291I&V302F, P291Q&S304F, P291Q&R292Q, P291L&S304F, P291Q&S304T, P291L&V303Q, V302H&V303F, R292Q&V303F, P291Q&V302F, P291L&V302F, V302Q&V303F, P291I&V303Q, V302Y&V303W, P291I&S304N, V302F&S304N, V302Q&V303W, K290L&P291L, P291Q&T207N, K290L&V303Q, K290L&V303W, K290I&P291L, R292Q&T207N, P291E&T207N, K290L&V303F, K290L&V303Y, K290L&V303I, K290I&V303F, K290I&V303Q, P291L&T207N, K290L&P291Q, K290E&V303F, K290E&R292Q, K290L&V302Q, K290L&R292Q, K290L&V303H, K290L&V302W, K290E&V303Q, K290E&V303Y, K290L&V302I, K290L&V302Y, K290E&V303H, K290E&V302Q, K290L&V302F, K290I&V302Q, K290I&V303W, K290E&P291L, Q295K, Q295R, Y296F, Y300F, P291I, P291L, P291V, Y296I, Y296V, S298F, S298H, S298N, S298T, S298W, S298Y, Y300F, Y300H, Y300I, Y300I, Y300L, Y300V, Y300V, Y300W, Y300W, R301H, R301W, V302F, V302H, V302I, V302L, V302Q, V302W, V302Y, V303F, V303H, V303I, V303L, V303Q, V303W, V303Y, S304F, S304H, S304N, S304T, S304W, S304Y, V305H, V305K, V305Q, V305R, V305W, or any combination thereof, wherein the numbering is according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution alters core-fucosylation at amino acid N297 as compared to the antibody Fc region without the substitution.

In one aspect, provided herein are antibody Fc regions comprising a substitution at one or more position(s) shown in a Table herein, wherein the numbering is according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution alters core-fucosylation at amino acid N297 as compared to the antibody Fc region without the substitution.

In one aspect, provided herein are antibody Fc regions comprising a substitution(s): K290, P291, R292, E293, E294, Q295, Y296, S298, Y300, R301, V302, V303, S304, V303, L306, T307, V308, L309, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

In one aspect, provided herein are antibody Fc regions comprising a substitution(s): P291E, P291K, P291Q, R292E, R292K, R292Q, Q295E, Y296E, Y296K, Y296Q, R301E, R301K, R301Q, P291I, V303F, V302F, P291L, S304N, S304T, S304F, V303W, V302W, R292Q, V303Q, V302H, V302Q, V302Y, K290L, T207N, K290I, V303Y, V303I, K290E, V303H, V302I, Q295K, Q295R, Y296F, Y300F, P291V, Y296I, Y296V, S298F, S298H, S298N, S298T, S298W, S298Y, Y300H, Y300I, Y300L, Y300V, Y300W, R301H, R301W, V302L, V303L, S304H, S304W, S304Y, V305H, V305K, V305Q, V305R, V305W, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

In one aspect, provided herein are antibody Fc regions comprising a substitution(s): P291E, P291K, P291Q, R292E, R292K, R292Q, Q295E, Y296E, Y296K, Y296Q, R301E, R301K, R301Q, P291I&V303F, V302F&V303F, P291Q&V303F, P291L&V303F, P291L&S304N, V303F&S304N, V303F&S304T, V303F&S304F, P291L&V303W, P291Q&S304N, P291I&V303W, V302W&V303F, V302W&V303W, V302F&V303W, P291I&V302F, P291Q&S304F, P291Q&R292Q, P291L&S304F, P291Q&S304T, P291L&V303Q, V302H&V303F, R292Q&V303F, P291Q&V302F, P291L&V302F, V302Q&V303F, P291I&V303Q, V302Y&V303W, P291I&S304N, V302F&S304N, V302Q&V303W, K290L&P291L, P291Q&T207N, K290L&V303Q, K290L&V303W, K290I&P291L, R292Q&T207N, P291E&T207N, K290L&V303F, K290L&V303Y, K290L&V303I, K290I&V303F, K290I&V303Q, P291L&T207N, K290L&P291Q, K290E&V303F, K290E&R292Q, K290L&V302Q, K290L&R292Q, K290L&V303H, K290L&V302W, K290E&V303Q, K290E&V303Y, K290L&V302I, K290L&V302Y, K290E&V303H, K290E&V302Q, K290L&V302F, K290I&V302Q, K290I&V303W, K290E&P291L, Q295K, Q295R, Y296F, Y300F, P291I, P291L, P291V, Y296I, Y296V, S298F, S298H, S298N, S298T, S298W, S298Y, Y300F, Y300H, Y300I, Y300I, Y300L, Y300V, Y300V, Y300W, Y300W, R301H, R301W, V302F, V302H, V302I, V302L, V302Q, V302W, V302Y, V303F, V303H, V303I, V303L, V303Q, V303W, V303Y, S304F, S304H, S304N, S304T, S304W, S304Y, V305H, V305K, V305Q, V305R, V305W, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

In one aspect, provided herein are antibody Fc regions comprising a deletion or substitution within 10 amino acids of a N297 glycosite, wherein: (a) the substitution(s) and/or deletion(s) are upstream of the glycosite comprising the removal of K, P, R, E, Q, or Y, or any combination thereof, (b) the substitution(s) and/or deletion(s) are downstream of the glycosite comprising the removal of S, Y, R, V, L, K, or T, or any combination thereof, (c) the substitution(s) and/or deletion(s) are upstream and/or downstream of the glycosite comprising the removal of K, P, R, E, Q, Y, S, V, L, or T, or any combination thereof, (d) the substitution(s) and/or deletions(s) comprise removal of K 7 positions upstream of the glycosite, P 6 positions upstream of the glycosite, R 5 positions upstream of the glycosite, E 4 positions upstream of the glycosite, E 3 positions upstream of the glycosite, Q 2 positions upstream of the glycosite, Y 1 positions upstream of the glycosite, S 1 positions downstream of the glycosite, Y 3 positions downstream of the glycosite, R 4 positions downstream of the glycosite, V 5 positions downstream of the glycosite, V 6 positions downstream of the glycosite, S 7 positions downstream of the glycosite, V 8 positions downstream of the glycosite, L 9 positions downstream of the glycosite, T 10 positions downstream of the glycosite, V 11 positions downstream of the glycosite, or L 112 positions downstream of the glycosite, or any combination thereof, (e) the substitution(s) and/or insertions(s) are upstream of the glycosite comprising the addition of E, K, Q, I, L, or V, or any combination thereof, (f) the substitution(s) and/or deletion(s) are downstream of the glycosite comprising the removal of E, K, Q, P, L, F, T, N, W, H, or Y, or any combination thereof, (g) the substitution(s) and/or deletion(s) are upstream and/or downstream of the glycosite comprising the removal of E, K, Q, P, I, V, L, F, T, N, W, H, or Y, or any combination thereof, and/or (h) the substitution(s) and/or insertion(s) comprise addition of I, L, E, K, or Q 7 positions upstream of the glycosite, I, L, V, E, K, or Q6 positions upstream of the glycosite, I, L, V, E, K, or Q 5 positions upstream of the glycosite, E, R or K 2 positions upstream of the glycosite, E, K, Q, F, I, L, V 1 position upstream of the glycosite, F, H, N, T, W, Y 1 position downstream of the glycosite, F, H, I, L, V, W 3 position downstream of the glycosite, E, K, Q, H, W 4 positions downstream of the glycosite, F, W, H, Q, Y, I, L 5 position downstream of the glycosite, F, W, H, Q, Y, I, L 6 position downstream of the glycosite, N, T, F, H, W, Y 6 position downstream of the glycosite, or N, H, K, Q, R, W 9 position downstream of the glycosite, or any combination thereof; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

In one aspect, provided herein are antibody Fc regions comprising one or more substitutions in Table 8, according to the EU numbering system; optionally wherein: the substitution alters a glycosylation feature as compared to the antibody Fc region without the substitution, the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution decreases core-fucosylation at amino acid N297 as compared to the antibody Fc region without the substitution.

In some embodiments, the antibody Fc region without the substitution comprises SEQ ID NO: 1. In some embodiments, the Fc region is a IgG1, IgG2, IgG3, or IgG4.

Further provided are antibodies comprising a Fc region provided herein.

Further provided are methods of treating a disease or condition in a subject in need thereof, the method comprising administering to the subject a composition comprising an antibody Fc region or the antibody comprising an Fc region described herein.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1UU show substitutions possible for glycosite-proximal amino acids, and the expected change in terms of relative preference for competing glycan features. Seq refers to sequence, struc refers to structure, ++ strong selection, + moderate selection, (+) weak selection, −− strong anti-selection, − moderate anti-selection, (−) weak anti-selection, where “selection” indicates that the substitution described in the row is consistent with the column header “glycan feature x is ‘preferred over’ or ‘selected over’ glycan feature y,” and anti-selection indicates the substitution is consistent with the opposite of the header “glycan feature y is preferred over glycan feature x”.

FIG. 2 is a bar graph showing the effect of a random Fc amino acid substitution on glycosylation pattern (Random), a Fc amino acid substitution based on sequence analysis (seq), a Fc amino acid substitution based on structural analysis (struct), as compared to the total glycosylation pattern (Total).

FIG. 3A shows the change in binding to FcRI when the Fc region of Rituximab is altered with a Y296R, S298K, or R301F substitution.

FIG. 3B shows the change in binding to FcRII when the Fc region of Rituximab is altered with a Q295E, S298K, or R301F substitution.

FIG. 3C shows the change in binding to FcRIII when the Fc region of Rituximab is altered with a S298K substitution.

FIGS. 4A-4B show binding of Rituximab variants to CD20 antigen as compared to wildtype Rituximab (Rituximab #14=Y296R, Rituximab #18=S298K, Rituximab #26=R301F, Rituximab #31=wildtype).

### DETAILED DESCRIPTION OF THE INVENTION

Glycosylation affects such protein properties as stability, solubility, half-life in the blood stream, interaction with their corresponding ligands, trafficking, etc. Usually, a certain glycosylation site on a protein can be occupied by a variety of glycan structures and the composition of a glycoprofile itself influences the protein properties as well. The importance of glycoengineering biologics, especially monoclonal antibodies, has been long recognized. Most therapeutic antibodies are immunoglobulin G (IgG) class. It has been shown previously that the type of glycans attached to the Fc-region of IgG influences its effector functions and the type of immune response triggered by the antibody.

Some of the most popular approaches for glycoengineering of therapeutic antibodies include glycosyltransferase knockout or overexpression, as well as media manipulation through precursors supplementation. However, in the experiments that involve introduction of point mutations in regions of the glycoprotein proximal to the glycosylation site it has been shown that the amino acid composition of a protein has an impact on the glycoprofile.

Table 1. Biantennary complex N-glycan (b14GlcNAc(-a16Fuc)-b14GlcNAc-b14Man(-b14GlcNAc)(-a1[3/6]Man-b12GlcNAc-b14Gal-a16Neu5Ac)) split over columns cross referenced with corresponding modulating mutations and modulated behaviors in the rows; predicted behaviors are shown in parentheses. IgG isoform, direction of change and citation are represented as: h-IgG1 (+), indicating human IgG1 shows an increase in glycosylation x. Behaviors associated with the presence of a glycosylation event appear below the double line such that: (+), indicates behavior x is positively influenced by the presence of the column glycan.

| | | | | b12GlcNAc | | |
|---|---|---|---|---|---|---|
| | | | -a1[3/6]Man | branching? | | |
| | | (-a16Fuc) | -this structure | More than 2 | | |
| | b14Glc | Core- | is a part of the | antennae are not | | |
| | Nac | fuco- | core, so should | really observed | b14Gal | a16Neu5Ac |
| | bisection | sylation | always be there | on IgG btw | galactosylation | sialylation |
| F241A | | | | | hIgG3(+)*; | hIgG3(+); |
| | | | | | hIgG1(+); | hIgG1(+); |
| | | | | | Rituximab*** (+) | Rituximab*** (+) |
| F243A | | | | | hIgG3(+)*; | hIgG3(+); |
| | | | | | hIgG1 (+) | hIgG1 (+) |
| V262E | | | | | hIgG1 (+) | hIgG1 (+) |
| V264E | | | | | hIgG1 (+) | hIgG1 (+) |
| V264A | | | | | hIgG3(+)* | hIgG3(+) |
| D265A | | | | | hIgG3(+)* | hIgG3(+) |
| Y296A | | | | | hIgG3(−)** | hIgG3(−) |
| Y296F | | | | | hIgG3(−)**** | hIgG3(−)**** |
| F296I | | | | mIgG1(+)[6] | mIgG1(−) | mIgG1(−) |
| | | | | hIgG1(+)[6] | | |
| R301A | | | | | hIgG3(+)* | hIgG3(+) |
| FcyRIIIa | | (−) | | (+)? | | (−) |
| binding | | | | | | |
| DC-SIGN | | | | | | (+) |
| binding | | | | | | |
| ADCC | | (−) | | | | |

*specifically, digalactosylation (structures with only 1 galactose on any of the 2 antennae are even downregulated)
**drastically reduced digalactosylation and monogalactosylation on the 2-3 arm
***Rituximab is a chimeric mouse/human therapeutic monoclonal IgG antibody
****Just one of the associated mutations, since the differences are observed between IGHG3*14 and IGHG311/12 allotypes, there are other amino acid substitutions that differ between these two amino acid sequences and they might have some impact too. I prioritized this particular mutation because it fits the observations of the impact of the position 296 on sialylation and galactosylation of Fc-linked IgG N-glycan reported in.

### Immunoglobulin G

Each IgG heavy chain carries an N-linked biantennary glycan covalently bound to Asn297 in the CH2 domain. These glycans are usually of a complex type and might contain galactose residues at the antennae, which in turn can be sialylated; core fucose and bisecting N-acetylglycosamine residues also might be present.

The structure of N-glycan bound to CH2 was shown to influence the affinity to IgG ligands. For instance, glycoforms lacking core fucose have increased affinity to FcyRIIIa and thus are thought to promote antibody-dependent cell-mediated cytotoxicity (ADCC), while presence of terminal sialylation reduces affinity to FcyRIIIa and increases affinity to the DC-SIGN receptor resulting in anti-inflammatory action. Thus, IgG N-glycosylation profile influences the course of immune response.

One of the many factors that define IgG N-glycome is the amino acid composition of the heavy chain. There is evidence that some IgG allotypes of the same subclass exhibit different profiles of Fc-linked N-glycosylation.

Human IgG3 variants expressed in CHO cells with amino acid residues interacting with the N-glycan substituted with Ala show changes in their N-glycome compared to the native IgG3 variant. In particular: replacement of residues FA241, FA243 (greatest increase in sialylation, 73% relative to the wild-type 4%), VA264, DA265, or RA301 with alanine resulted in increased galactosylation and sialylation relative to the wild-type oligosaccharide chains. YA296 leads to severely decreased sialylation (around 0%).

Human IgG1: site-directed mutations disrupting the protein-carbohydrate interface (F241A, F243A, V262E, and V264E) increased galactosylation and sialylation of the Fc and, concomitantly, reduced the affinity for FcγRIIIA. Their data also indicates that destabilization of the glycan-protein interactions, rather than increased galactosylation and sialylation, modifies the Fc conformation(s) relevant for FcγR binding.

Mutations of Y407 in the CH3 domain of hinge-deleted IgG4 and IgG1 significantly increase sialylation, galactosylation, and branching of the N-linked glycans in the CH2 domain. These mutations also promote the formation of monomeric assemblies (one heavy-light chain pair).

Human IgG3 variants expressed in HEK cells differ in the levels of galactosylation and bisection in their Fc-linked N-glycomes. Between donors, however, the IGHG3*14 allotype seems to exhibit a higher degree of galactosylation and sialylation in comparison with IGHG3*11/12 allotypes.

Missense mutations in the ighg1, ighg2b and Ighg2c gene are among the candidate SNPs discovered by QTL analysis of IgG N-glycome in the Collaborative Cross (CC) inbred mouse strains. Unpublished QTL LC-MS analysis of IgG1 N-glycosylation in a CC cohort also lists a missense mutation rs51376262 in ighg1 as candidate associated with changes in IgG N-glycan profile. The candidate missense mutation leads to a F296I substitution (numbering according to the human homolog). This mutation is present in ighg1 allele derived from C57BL/6 and NOD mice and is associated with higher ratio of agalactosylated glycoforms and lower abundance of digalactosylated and sialylated glycans. Moreover, C57BL/6 and CD1 mice expressing IgG1 variant characterized by F296I substitution have significantly lower levels of IgG1 sialylation and digalactosylation than strains expressing IgG1 variant with F296.

### Other Proteins

Protein disulfide isomerase (PDI), an endoplasmatic reticulum enzyme that catalyzes formation/disruption of disulfide bonds. Point mutations introduced to the amino acid positions that are supposed to interact with a proximal glycan influence the PDI glycoprofile. For instance, Y178A resulted in increase in core-fucosylation; Y178F in reduction in complex glycans.

Rattus norvegicus cluster of differentiation 2 adhesiondomain (CD2ad). The wild-type sequence surrounding the glycosylation site is Leu63Ala64Asn65Gly66Thr67, with Leu at n−2 (CD2-L). Mutated Leu63→Hist/Phe changed the glycoprofile. Phe63 has more hybrid structures and fewer complex profile as compared to wild type, Hist63 has an intermediate profile.

Fibroblast growth factor 9 (FGF9). Glycan composition of FGF9-A (with Ala at n−2, n being the position of glycosylated Asn residue) and FGF9-F (with Phe at n−2) differ in that the latter exhibits more hybrid structures.

### High-Confidence Glycosite-Proximal Mutations and Corresponding Differential Glycosylation

Human/mouse IgG: the amino acid residue in position n−1 from the glycosylated N supposedly interacts with the core-fucose of the N-glycan and the type of amino acid in this position affects the Fc-N-glycosylation profile. In mice, the amino acid substitution F296I in the CH2 domain of IgG1 leads to decreased sialylation and galactosylation. In human IgG3 allotypes IGHG3*11 and 12 with Y296F substitution also exhibit lower galactosylation and sialylation. Introduction of a point mutation Y296A in human IgG3 sequence lead to drastic reduction of sialylation in CHO cells. One of the possible explanations for the impact of this substitution is that the changes in the interaction between the peptide backbone and core-fucose of the glycan lead to the glycan being inaccessible for glycosyltransferases that modify the antennae.

Human IgG: substitution Y407E in the CH3 domain leads to increased galactosylation and sialylation of IgG Fc.

Data obtained on rat CD2ad and human FGF9 suggests that Leu (n−2) Phe substitution leads to increase in hybrid glycans at Asn (n) and reduction in complex glycans.

TABLE 2
Amino acid differences between IgG3 allotypes IGHG3*14 and
IGHG3*11/12 that differ in Fc-linked N-glycoprofiles.

| | amino | substitution (IGHG3*14 −> |
|---|---|---|
| rs | acid # | IGHG3*11/12) |
| rs74093865 | 291 | P −> L |
| rs12890621 | 296 | Y −> F |
| rs77307099 | 384 | S −> N |
| rs78376194 | 384 | S −> N |
| rs1051112 | 436 | F −> Y |

### Non-Limiting Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some embodiments, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### Fc Regions and Antibodies

In one aspect, provided herein are Fc regions and antibodies comprising Fc regions. In some embodiments, Fc regions and antibodies of this disclosure have an increased or decreased effector function as compared to a human IgG (e.g., SEQ ID NO: 1). In some embodiments, Fc regions and antibodies of this disclosure have a distinct effector function as compared to a human IgG (e.g., SEQ ID NO: 1). Effector function refers to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of a target cells in the presence of complement, where the complement action pathway is initiated by the binding of C1q to antibody bound with the target.

In some embodiments, provided herein are Fc regions and antibodies characterized by exhibiting ADCC that is reduced by at least about 30%, at least about 40%, at least about 50%, at least about 60%, at least about 70% or more as compared to an Fc region or antibody comprising a non-variant Fc region, i.e., an antibody with the same sequence identity but for the substitution(s) that decrease ADCC (such as human IgG1, SEQ ID NO: 1). In some embodiments, the disclosure provides antibodies comprising Fc regions characterized by exhibiting CDC that is reduced by at least about 30%, at least about 40%, at least about 50%, at least about 60%, at least about 70% or more as compared to an Fc region or antibody comprising a non-variant Fc region, i.e., an antibody with the same sequence identity but for the substitution(s) that decrease CDC (such as human IgG1, SEQ ID NO: 1). In some embodiments, the disclosure provides antibodies comprising Fc regions characterized by exhibiting ADCP that is reduced by at least about 30%, at least about 40%, at least about 50%, at least about 60%, at least about 70% or more as compared to an Fc region or antibody comprising a non-variant Fc region, i.e., an antibody with the same sequence identity but for the substitution(s) that decrease ADCP (such as human IgG1, SEQ ID NO: 1). In certain embodiments, the antibodies of this disclosure have reduced effector function as compared with human IgG1.

In some embodiments, provided herein are Fc regions and antibodies characterized by exhibiting ADCC that is reduced by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region, i.e., an antibody with the same sequence identity but for the substitution(s) that decrease ADCC (such as human IgG1, SEQ ID NO: 1). In some embodiments, the disclosure provides antibodies comprising Fc regions characterized by exhibiting CDC that is reduced by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region, i.e., an antibody with the same sequence identity but for the substitution(s) that decrease CDC (such as human IgG1, SEQ ID NO: 1). In some embodiments, the disclosure provides antibodies comprising Fc regions characterized by exhibiting ADCP that is reduced by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region, i.e., an antibody with the same sequence identity but for the substitution(s) that decrease ADCP (such as human IgG1, SEQ ID NO: 1). In certain embodiments, the antibodies of this disclosure have reduced effector function as compared with human IgG1.

In some embodiments, provided herein are Fc regions and antibodies characterized by exhibiting ADCC that is increased by at least about 30%, at least about 40%, at least about 50%, at least about 60%, at least about 70% or more as compared to an Fc region or antibody comprising a non-variant Fc region, i.e., an antibody with the same sequence identity but for the substitution(s) that increase ADCC (such as human IgG1, SEQ ID NO: 1). In some embodiments, the disclosure provides antibodies comprising Fc regions characterized by exhibiting CDC that is increased by at least about 30%, at least about 40%, at least about 50%, at least about 60%, at least about 70% or more as compared to an Fc region or antibody comprising a non-variant Fc region, i.e., an antibody with the same sequence identity but for the substitution(s) that increase CDC (such as human IgG1, SEQ ID NO: 1). In some embodiments, the disclosure provides antibodies comprising Fc regions characterized by exhibiting ADCP that is increased by at least about 30%, at least about 40%, at least about 50%, at least about 60%, at least about 70% or more as compared to an Fc region or antibody comprising a non-variant Fc region, i.e., an antibody with the same sequence identity but for the substitution(s) that increase ADCP (such as human IgG1, SEQ ID NO: 1). In certain embodiments, the antibodies of this disclosure have increased effector function as compared with human IgG1.

In some embodiments, provided herein are Fc regions and antibodies characterized by exhibiting ADCC that is increased by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region, i.e., an antibody with the same sequence identity but for the substitution(s) that increase ADCC (such as human IgG1, SEQ ID NO: 1). In some embodiments, the disclosure provides antibodies comprising Fc regions characterized by exhibiting CDC that is increased by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region, i.e., an antibody with the same sequence identity but for the substitution(s) that increase CDC (such as human IgG1, SEQ ID NO: 1). In some embodiments, the disclosure provides antibodies comprising Fc regions characterized by exhibiting ADCP that is increased by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region, i.e., an antibody with the same sequence identity but for the substitution(s) that increase ADCP (such as human IgG1, SEQ ID NO: 1). In certain embodiments, the antibodies of this disclosure have increased effector function as compared with human IgG1.

Non-limiting examples of Fc mutations in IgG1 that may change ADCC, CDC, and/or ADCP include substitutions at one or more of positions: K290, P291, R292, E293, E294, Q295, Y296, S298, Y300, R301, V302, V303, S304, V303, L306, K290, T307, V308, L309 V305, where the numbering system of the constant region is that of the EU index as set forth by EU. In certain embodiments, the antibodies of this disclosure have reduced effector function as compared with human IgG1. Additional, non-limiting examples are provided in the claims and Tables herein.

In some embodiments, provided herein are Fc regions and antibodies having reduced fucosylation as compared to an Fc region or antibody comprising a non-variant Fc region, i.e., an antibody with the same sequence identity but for the substitution(s) that decrease fucosylation (e.g., as compare to human IgG). In some embodiments, the reduction is by at least about 30%, at least about 40%, at least about 50%, at least about 60%, at least about 70% or more as compared to an Fc region or antibody comprising a non-variant Fc region. In some embodiments, the reduction is by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region. In some embodiments, the fucosylation is reduced at position N297, set forth using the EU numbering scheme.

In some embodiments, provided herein are Fc regions and antibodies having increased fucosylation as compared to an Fc region or antibody comprising a non-variant Fc region, i.e., an antibody with the same sequence identity but for the substitution(s) that increase fucosylation (e.g., as compare to human IgG). In some embodiments, the increase is by at least about 30%, at least about 40%, at least about 50%, at least about 60%, at least about 70% or more as compared to an Fc region or antibody comprising a non-variant Fc region. In some embodiments, the increase is by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region. In some embodiments, the fucosylation is increased at position N297, set forth using the EU numbering scheme.

In some embodiments, provided herein are Fc regions and antibodies having an increase or decrease in a glycosylation feature as compared to an Fc region or antibody comprising a non-variant Fc region, i.e., an antibody with the same sequence identity but for the substitution(s) that change glycosylation (e.g., as compare to human IgG). In some embodiments, the change is by at least about 30%, at least about 40%, at least about 50%, at least about 60%, at least about 70% or more as compared to an Fc region or antibody comprising a non-variant Fc region. In some embodiments, the change is by at least about In some embodiments, the reduction is by at least about 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, or 100-fold (e.g., about 2-fold to about 100-fold) as compared to an Fc region or antibody comprising a non-variant Fc region. In some embodiments, the glycosylation is changed at position N297, set forth using the EU numbering scheme. Non-limiting example glycosylation features are provided herein. For instance, glycosylation features and associated functions are shown in Table 6 and Table 7.

In the context of IgG antibodies, the IgG isotypes each have three CH regions. Accordingly, “CH” domains in the context of IgG are as follows: “CHI” refers to positions 118-220 according to the EU index as in Kabat. “CH2” refers to positions 237-340 according to the EU index as in Kabat, and “CH3” refers to positions 341-447 according to the EU index as in Kabat. For instance, SEQ ID NO: 1 according to the EU index begins at position 118, and residues PREEQYNSTYRVVSVLT correspond to positions 291 to 307.

TABLE 6
Biological roles and therapeutic potential of glycan epitopes

| Modification type | Actions | Context | Therapeutic importance |
|---|---|---|---|
| Core-fucosylation | decreased affinity to | Fc domain of IgG | Not desirable for mAbs used to |
| | activating FcγRIIIA and | | treat cancer (achieved by |
| | FcγRIIIB, decreased ADCC | | knocking out FUT8); desirable for |
| | | | anti-inflammatory properties of |
| | | | IVIG |
| | | α-fetoprotein | overrepresented in cancers, an |
| | | | approved biomarker of |
| | | | hepatocellular carcinoma |
| | | epidermal growth | associated with malignancy in |
| | | factor receptor (EGFR) | breast cancer |
| α1-3-fucosylation of | | blood plasma proteins | overrepresented in cancers, an |
| triantennary glycans | | | approved biomarker of |
| | | | hepatocellular carcinoma |
| α1-3-core | | present in plant and | immunogenic, hence the need to |
| fucosylation | | insect cells, absent in | glycoengineer cell cultures |
| | | humans | producing therapeutic |
| | | | glycoproteins |
| Sialylation | increases protein solubility | any, both on N- and O- | sialylation is beneficial for the |
| | and half-life in bloodstream, | glycans | stability of the therapeutic |
| | inhibit proteolytic cleavage | | proteins |
| | and excretion | | |
| | recognized by complement, | believed to act as such | increased sialylation is a cancer |
| | siglecs and other immune- | in general, a concrete | marker; cancer treatment target |
| | related receptors to inhibit | example is that α2-6 | |
| | innate immune responses, | sialylation of the | |
| | induces immune tolerance, | TNFR1 death receptor | |
| | promotes an apoptosis- | inhibits apoptosis | |
| | resistant phenotype in | | |
| | cancers | | |
| | lowers affinity for activating | IgG | anti-inflammatory action of IVIG, |
| | FcγRs, increases binding to | | reduction of immunogenicity |
| | various lectin receptors | | |
| | suppress angiogenesis in | Vascular endothelial | important for anti-VEGF cancer |
| | tumors | growth factor (VEGF) | therapy |
| | stimulates tumor cell | collagen-selective | cancer marker, therapeutic target |
| | migration and invasion | integrins, α2-6 | |
| | | sialylation, | |
| | | polysialylation | |
| Sialyl Lewis | interact with selectins, | O-glycans | cancer marker, therapeutic target - |
| structures | activate receptor tyrosine | | anti-sialyl Lewis A antibody; |
| | kinases, increase tumor cell | | useful modification for homing of |
| | adhesion and aggregation, | | the therapeutic CAR T-cells |
| | promote metastasis | | |
| Sialyl Tn antigen | | truncated O-glycans | cancer marker, therapeutic target - |
| | | | vaccine did not pass clinical |
| | | | trials |
| N-glycolyl | non-human sialic acid | any | immunogenic for humans; |
| neuraminic acid | | | glycoengineering of cell cultures |
| | | | used for production of therapeutic |
| | | | proteins; cancer marker |
| Bisection of | associated with pro- | Fc domain of IgG | glycoengineering of cell cultures |
| complex N-glycans | inflammatory action; | | to increase ADCC potency of |
| | prevents further glucan | | mAbs |
| | modifications: branching | | |
| | and core-fucosylation | | |
| | suppresses further | any | cancer suppression through |
| | processing and elongation of | | blocking of branching |
| | N-glycans (branching and | | |
| | consecutive sialylation), | | |
| | hence, anti-cancer potential | | |
| Branching of | controls the threshold of | T-cells | potential target of autoimmunity |
| complex N-glycans | activation and autoimmunity | | treatments |
| | (lowered in absence) | | |
| | branches could be further | any (integrins in | cancer marker, potential |
| | modified and capped with | particular, E-cadherin, | therapeutic target (through |
| | sialic acids, leads loss of | EGFR) | increased MGAT3 activity and |
| | contact inhibition, increased | | elevated bisection which prevents |
| | cell motility and tumour | | branching) |
| | formation, enhanced | | |
| | invasion and metastasis | | |
| Galactosylation | required for addition of | Fc domain of IgG | glycoengineering of cell cultures |
| | terminal sialic acids; | | used for production of therapeutic |
| | initiates anti-inflammatory | | mAbs with increased sialylation |
| | signaling; however, | | |
| | complement activation | | |
| | through C1q binding is | | |
| | reported also | | |
| alpha-1,3-galactose | present in mammals except | any | Immunogenic for humans, |
| | for primates | | prevents xenotransplantation |
| Oligomannose N- | enhanced clearance from the | any | Elevated in cancers |
| glycans | circulation via interaction | | |
| | with ManR | | |
| | enhanced clearance, | IgG | undesirable modification of |
| | promotes ADCC, decreased | | therapeutic mAbs |
| | affinity for complement | | |
| β-1,2-xylose | Plant glycan | any | Immunogenic for humans |

TABLE 7
Examples of Ligand-Receptor Interactions Modified by Glycan

| | | | | Alt. | Glycan |
|---|---|---|---|---|---|
| Mechanism | Ligand | Receptor | Type | Glycosylation | Impact |
| Co-reception | Delta and | Notch EGF | O | Presence/absence | Boundary |
| | Serrate | motifs | | of glycan | formation, T |
| | | | | | cell and |
| | | | | | marginal zone |
| | | | | | B cell |
| | | | | | development |
| Co-reception | CD48 | 2B4 | N and O | Differential | Proper |
| | | (CD244) | | sialylation | glycosylation |
| | | | | | necessary for |
| | | | | | binding |
| Co-reception | NMDA | NMDAR | N | Differential | Binding |
| | | | | mannosylation | affinity |
| Structural | Antigen | IgG1 | N | Core | Antibody- |
| | | | | fucosylation | dependent cell- |
| | | | | | mediated |
| | | | | | cytotoxicity |
| Structural | Fibronectin | Integrin | N | Removal of | Integrin |
| | | | | glycosylation | assembly and |
| | | | | sites | activity |
| Structural | ACE2 | SARS- | N | Maturation of | Destabilization |
| | | CoV-2 | | oligomannose | of the spike |
| | | spike | | | RBD |
| Ligand | VEGF-C | VEGFR3 | GAG | Differential | Inhibition of |
| Guiding | | | | sulfation on | Lymphogenesis |
| | | | | heparin sulfate | |
| Ligand | BMP, FGF | Smad1/5, | GAG | Differential | Cartilage |
| Guiding | | Erk1/2 | | sulfation | degradation |
| | | | | | and repair |
| Multiple | Multiple | NCAM | N | Differential | Cerebellum |
| | | | | polysialylation | formation and |
| | | | | | glioblastoma |
| | | | | | migration |

### Glycosylation Features

Fc regions and antibodies comprising Fc regions as described herein may comprise one or more glycosylation features or glycans. A glycosylation feature may comprise one or more monosaccharides linked glycosidically. A glycosylation feature may be present or otherwise associated with the Fc region. The association may comprise one or more covalent (e.g., glycosidic) bonds or the association may be non-covalent. A glycosylation feature may comprise any number of monosaccharides or derivatives. A glycosylation feature may comprise 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more monosaccharides or derivatives thereof.

Glycosylation features as described herein may comprise any monosaccharide or derivative thereof. Monosaccharides may comprise D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (Kdo), D-galacturonic acid (GalA), L-iduronic acid (IdoA), L-rhamnose (Rha), L-fucose (Fuc), D-xylose (Xyl), D-ribose (Rib), L-arabinofuranose (Araf), D-glucuronic acid (GlcA), D-allose (All), D-apiose (Api), D-fructofuranose (Fruf), ascarylose (Asc), ribitol (Rbo). Derivatives of monosaccharides may comprise sugar alcohols, amino sugars, uronic acids, ulosonic acids, aldonic acids, aldaric acids, sulfosugars, or any combination or modification thereof. A sugar modification may comprise one or more of acetylation, propylation, formylation, phosphorylation, or sulfonation or addition of one or more of deacetylated N-acetyl (N), phosphoethanolamine (Pe), inositol (In), methyl (Me), N-acetyl (NAc), O-acetyl (Ac), phosphate (P), phosphocholine (Pc), pyruvate (Pyr), sulfate(S), sulfide (Sh), aminoethylphosphonate (Ep), deoxy (d), carboxylic acid (-oic), amine (-amine), amide (-amide), ketone (-one). Such modifications may be present at any position on the sugar, as designated by standard sugar naming/notation. In some embodiments, at least 1, 2, 3, 4, 5, 6, 7, 8, 9, 10 or more modifications are present on the monosaccharide. In some embodiments, no more than 10, 9, 8, 7, 6, 5, 4, 3, 2, 1, or fewer modifications are present on the monosaccharide. Monosaccharides may comprise any number of carbon atoms. Monosaccharides may comprise any stereoisomer, epimer, enantiomer, or anomer. In some embodiments, monosaccharides comprise 3, 4, 5, 6, 7, 8, 9, 10, 11, 12, 13, 14, 15, or more carbon atoms.

In some embodiments, a glycosylation feature may comprise glyceraldehyle, threose, erythrose, lyxose, xylose (Xyl), arabinose, ribose, talose, galactose (Gal), idose, gulose, mannose (Man), glucose (Glc), altrose, allose, sedoheptulose, mannoheptulose, N-acetyl-galactosamine (Glc2NAc), glucuronic acid (GlcA), 3-O-sulfogalactose (Gal3S), N-acetylneuraminic acid (Neu5Ac), 2-keto-3-deoxynonic acid (Kdn), or any combination thereof.

A glycosylation feature may comprise one monosaccharide. A glycosylation feature may comprise a plurality of monosaccharides. In such cases, the monosaccharides may be connected in any configuration through any suitable glycosidic bond(s). Glycosidic bonds between monosaccharides in a polysaccharide glycosylation feature may be alpha or beta and connect any two carbon atoms between adjacent monosaccharide residues through an oxygen atom. In some embodiments, the glycosylation feature of glycan is an N-linked, O-linked, C-linked, or S-linked glycan. In some embodiments, more than one glycosylation feature is present on a single biomolecule. The more than one glycosylation features may all be linked in the same manner (e.g., N-linked, O-linked, C-linked, S-linked), or they may be independently N-linked, O-linked, C-linked, or S-linked. Glycosylation features may be branched, linear, or both. Glycosylation features may be biantennary, triantennary, tetra-antennary, or any combination thereof. In some embodiments, the glycosylation feature comprises a polysaccharide epitope. In some embodiments, the glycosylation feature comprises high-mannose. In some embodiments, the glycosylation feature comprises sialylation. In some embodiments, the glycosylation feature comprises fucosylation. In some embodiments, the glycosylation feature comprises hybrid, complex, core or distally fucosylated, terminally sialylated, terminally galactosylated, terminally GlcNAc-ylated, GlcNAc-bisected, or poly-sialylated.

A glycosylation feature may be described in relative terms. A glycosylation feature may be described as increased or decreased with respect to the amount of a given monosaccharide in the glycosylation feature relative to a reference glycosylation feature. For example, a glycosylation feature may be described as an increase or increased in sialylation or fucosylation if the glycosylation feature comprises more sialic acid or fucose residues, respectively, than a reference glycan. Alternatively or additionally, a glycosylation feature may be described as increased or decreased with respect to the configuration (e.g., branched, linear, biantennary, tri-antennary, tetra-antennary, penta-antennary) of the glycosylation feature relative to a reference glycosylation feature. For example, a glycosylation feature may be described as an increase or increased in branching if the glycosylation feature comprises more branches than a reference glycosylation feature. In some embodiments, a glycosylation feature may be described as increased or decreased in one or more of high-mannose, sialylation, fucosylation, hybrid, complexity, core or distally fucosylation, terminal sialylation, terminal galactosylation, terminal GlcNAc-ylation, GlcNAc-bisection, or poly-sialylation.

### Fucosylation

In some embodiments, a glycosylation feature comprises fucosylation. In some embodiments, the fucosylation is core-fucosylation. N-glycans attached to immunoglobulin G almost exclusively contain a1,6-linked core-fucose. Core-fucosylation on Fc-linked IgG N-glycans is considered anti-inflammatory and has the strongest evidence supporting suggested functions of all IgG N-glycan traits. Core-fucosylation of Fc-linked IgG N-glycans leads to decreased affinity of IgG to the activating FcγRIIIA and FcγRIIIB and, therefore, dampens antibody-dependent cell-mediated cytotoxicity (ADCC). This makes core fucose a non-desirable modification for the therapeutic monoclonal Abs that are used in cancer treatment and therefore are required to efficiently induce ADCC of cancer cells. Usually this is achieved by knocking out α-1,6-fucosyltransferase (FUT8) that adds core fucose. However, core-fucosylation has different functions on different proteins. The FUT8 enzyme is known to be overexpressed in cancers. In particular, core fucosylation of α-fetoprotein is an approved biomarker for the early detection of hepatocellular carcinoma (HCC), that allows to distinguish it from chronic hepatitis and liver cirrhosis. Increased core fucosylation of epidermal growth factor receptor (EGFR) was associated with malignancy in breast cancer. Core hyperfucosylation and increase in α1-3-fucosylation of triantennary glycans on blood plasma proteins is observed in hepatocellular carcinoma and a diagnostic test for liver health based on blood plasma glycan profiling was launched.

As concerns O-glycans, the characteristics of fucosylation of Lewis antigen define Lewis blood groups. Lea contains α-1,4-fucose (added by FUT3 enzyme) and Leb contains both α-1,4- and α-1,2-fucose residues (added by FUT2). Lewis blood groups define susceptibility to certain bacterial and viral pathogens, but are clinically insignificant for blood transfusion or in pregnancy. Increasing the incidence of fucosylation to introduce sLex antigens on chimeric antigen receptor T-cells (CAR-T) that are used in cancer therapy to enhance their homing to targets may be performed using the methods herein.

### Sialylation

In some embodiments, a glycosylation feature comprises sialylation. Sialic acids are often terminal modifications of N- and O-glycans. They are negatively charged at physiological pH and therefore in general increase protein solubility and inhibit proteolytic cleavage. Negative charge is important, for instance, sialylation of glycans attached to erythropoietin reduces its binding to its receptors due to electrostatics, and renal clearance is also reduced due to electrostatic repulsion from the negatively charged glomerulus. Increased terminal sialylation increases the serum half-life of glycoproteins, both on O- and N-glycans, therefore in some cases it is a desirable modification to increase the half-life of various therapeutic proteins.

A-2,6-linked sialylation also is important for anchoring of the membrane receptors on the cell surface through a galectin-dependent mechanism. Receptor a2-6 sialylation causes release from the galectin lattice, leading to receptor internalization. Conversely, a2-6 sialylation can facilitate the surface retention of other types of receptors.

Sialylation influences conformation of several proteins: $1 integrin; clustering of CD45, EGFR, and PECAM; and cell surface retention of PECAM and the Fas death receptor interaction between CD8 and MHC class I proteins.

Cell surface sialylation is recognized by complement, siglecs and other immune-related receptors to inhibit innate immune responses in the central nervous system. Sialic acids are ligands to such lectins as, for example, CD22 and Siglec-G, that inhibit BCR signaling and promote immune tolerance. α2-6-linked sialic acids may confer an apoptosis-resistant phenotype, since they prevent binding of apoptosis-inducing galectins that recognize terminal galactose residues. On the other hand, ST6Gal-I-mediated α2-6 sialylation of the TNFR1 death receptor inhibits TNFα directed apoptosis in macrophages.

Sialylation of immunoglobulin G glycans: the generally accepted consensus is that terminal α2-6-sialyaltion of Fc-linked IgG N-glycans is anti-inflammatory, although there is some contradictory evidence. Sialylation is thought to be responsible for the anti-inflammatory activity of intravenous immunoglobulin (IVIg). Fc-linked sialylated N-glycans are thought to decrease inflammation through lower affinity for activating FcγRs, binding to various lectin receptors (dendritic cell-specific intercellular adhesion molecule grabbing non-integrin, C-type lectin domain family 4 member A, B-cell receptor CD22). At the same time, there are reports of increased binding of sialylated IgG to the C1q binding and subsequent proinflammatory action. A system for production of heavily sialylated antibodies has been described with an IgG1 having an introduced F243A mutation being co-expressed with the human 2,6-sialyltransferase 1 (ST6GAL1) and β1,4-galactosyltransferase 1 (B4GALT1) in CHO cells.

In cancer sialylation is elevated and contributes to tumor evasion of immune response through interaction with siglecs. In particular, expression of all three types of polysialyltransferases that add α2-8 linked sialic acid residues is enhanced in tumors, while sialidases seem to be down regulated. Polysialylation is associated with invasiveness and poor clinical outcome in a number of cancers.

A2-6-sialylation of collagen-selective integrins (β1 integrin, for example), which is additionally enhanced by upregulation of branching N-glycan structures, stimulates tumor cell migration and invasion.

On the other hand, sialylation of glycans on the VEGF (Vascular endothelial growth factor) prevents its interaction with galectin 1 that recognizes terminal galactose residues and suppresses angiogenesis in tumors which is important for the efficacy of anti-VEGF treatment.

As for O-glycans, the sialyl Thomsen-nouvelle antigen (sialyl Tn) is a well-known cancer marker, almost absent from normal epithelial cells. However, particular proteins carrying it are not yet all identified, among them are: CD44 (adhesion protein); mucin Muc 1 in breast and gastric cancer cells; β1 integrin and osteopontin in murine cancer cells.

Sialyl Lewis (sLe) structures (SLex and Slea, specifically, containing a2-3-linked sialic acid residues) are upregulated on the tumor cell surface and promote tumor cell adhesion and metastasis to the endothelium through interaction with endothelial selectins. sLe structures are also recognized by siglecs and thus contribute to immune escape in cancer. Sialyl Lewis epitopes can also lead to invasive cancer phenotype through the hyperactivation of the receptor tyrosine kinases. Slea, also referred to as CA19-9, is a marker of digestive system cancers, although it cannot be used in individuals who are Lewis antigen-negative. Antibodies to CA19-9 are considered as anti-cancer treatment. SLex is the well-known ligand for selectins and thus is involved in promotion of metastasis.

Therapeutic usage of sialylation includes glycoengineering of therapeutic proteins to reduce immunogenicity, e.g., production of Fab-sialylated mAbs. Increased sialylation of IVIG enhances its potency. Glycoengineering of sialylated natural killer cells is a strategy to direct them to CD22-expressing cells of B-cell lymphoma. CAR T-cells that are used in cancer therapy are sLex-glycoengineered to facilitate their homing to target tissues. Since increased sialylation is a hallmark of many cancers that promotes immune evasion, metastasis, and more aggressive tumor phenotypes it is also a popular target for anti-cancer therapeutic approaches, such as desialylation via sialidase conjugate delivery to tumors, introduction of glycomimetics that block the interaction between sialic acids and selectins or siglecs, or antibodies against specific glycan epitopes containing sialic acid residues.

In contrast to other mammals, humans are incapable of synthesizing N-glycolylneuraminic acid due to a mutation that rendered the CMAH enzyme inactive and therefore human glycans are sialylated with N-acetylneuraminic acid residues. Humans, however, receive N-glycolylneuraminic acid from food and it is found incorporated in glycans of some human tumors, where it apart from promoting cancer by mechanisms described above, also induces immune response. Therapeutic mAbs produced in non-human cell cultures also can contain N-glycolyl neuraminic acid residues and thus elicit immune response. Glycoengineering is required to exclude this negative effect.

### Bisection

In some embodiments, a glycosylation feature comprises bisection. Bisecting N-acetylglucosamine is a modification of N-glycans that is introduced by the beta-1,4-mannosyl-glycoprotein 4-beta-N-Acetylglucosaminyltransferase (MGAT3). Bisection suppresses further processing and elongation of N-glycans such as the β1,6 branching structures and core-fucosylation.

Bisection of Fc-linked IgG N-glycans is associated with inflammation and ADCC. The observed association could arise due to that lower core-fucosylation usually being accompanied by increased bisection. Increased bisection of IgG N-glycans in autoimmune diseases such as systemic lupus erythematous and rheumatoid arthritis might be due to elevated levels of Fab-linked glycans in autoimmune diseases that are known to be more processed than the Fc-linked structures. However, GnTIII cDNA transfected CHO cell line was created to obtain IgG with increased bisection and elevated ADCC, although the effect is likely indirect.

Since suppression of branching of N-glycans by bisection leads to cancer suppression, enhancing activity of MGAT3 is a potential anti-cancer therapeutic strategy.

### Galactosylation

In some embodiments, a glycosylation feature comprises galactosylation. In some cases, antennary 2,6-galactosylation of Fc-linked IgG glycans is anti-inflammatory. In many autoimmune, inflammatory, infectious diseases and cancers the abundance of this modification is decreased which is regarded as evidence for proinflammatory activity of agalactosylated IgG glycoforms. In some cases, galactosylated IgG1 in the immune complexes is necessary to initiate anti-inflammatory signaling through the inhibitory receptor FcγRIIB and binding of IgG1 for the FcγRII2b in mice. Moreover, galactosylation is a prerequisite for antennary sialylation, which is believed to be anti-inflammatory. At the same time, galactosylated IgG glycoforms can act in pro-inflammatory manner: activate complement through C1q binding, enhance ADCC through activating FcγRs.

α-Gal epitope (galactose-α-1,3-galactose) is another major immunogenic glycan structure. Primates, including humans, are unable to synthesize this structure. At the same time, therapeutic proteins produced in non-human cell lines may contain it and induce immune response. α-Gal is also one of the most important antigens that prevents xenotransplantation.

### Branching N-Glycans

In some embodiments, a glycosylation feature comprises branching N-glycans. Decreased branching in T-cells leads to lower threshold of activation and autoimmunity, decreased branching of MHCII leads to decreasing carbohydrate antigen presentation by MHC class II and leading to loss of T cell stimulatory activity.

Increased branching of both N- and O-glycans is one of the cancer hallmarks. Increased branching of N-glycans in cancers is due to elevated MGAT5 activity and resulted in loss of contact inhibition, increased cell motility and tumour formation, enhanced invasion and metastasis. Branched structures can be further elongated with poly-N-acetyllactosamine and capped with sialic acids and antennary fucose residues. Such structures are promoting tumor growth and metastasis via engagement of galectin receptors. For example, abundant branching glycans on integrins are a cancer marker associated with metastasis, branched glycans on E-cadherin disrupt cell adhesion and contribute to tumour invasiveness and metastases, branched glycans on EGFR promote cancer.

### Truncated Glycans in Cancer

In some embodiments, a glycosylation feature comprises truncated glycans. Synthesis of incomplete glycan structures is a common feature of early stages of cancer, for example, elevated levels of truncated O glycans such as the disaccharide Thomsen-Friedenreich antigen (T antigen, also known as core 1) and the monosaccharide GalNAc (Tn antigen) and their sialylated forms (ST and STn, respectively), which result from the incomplete synthesis of O-glycans. There are examples of vaccines developed that target mucin-related Tn, STn, and T antigens for suppression of breast cancer.

### Oligomannose Type N-Glycans

In some embodiments, a glycosylation feature comprises oligomannose. Prescence of oligomannose glycans usually shortens the half-life of proteins in the blood stream because these glycans are recognized by the mannose receptor and removed from circulation, so it can be an unfavorable modification for therapeutic glycoproteins. Immunoglobulin G with high-mannose glycans linked to Fc domain was shown to efficiently induce ADCC (probably, due to absence of core-fucose on this type of glycans) but fail to fix complement. High-mannose glycans are also elevated in cancers and are considered an example of incomplete and impaired glycan synthesis in tumor cells.

### Immunogenic Glycans in Humans

In some embodiments, a glycosylation feature comprises immunogenic glycan(s). Alpha-1,3-galactose and α-galactose (α-Gal) and N-glycolylneuraminic acid addition if produced in CHO or mouse cells can induce immune response; plant cells can add core α-1,3-fucose and β-1,2-xylose, while insect cells can introduce core α-1,3-fucose. Cetuximab, mouse-human chimeric IgG1 mAb produced in a murine cell line, is known to induce allergic reactions due to α-1,3-galactose presence. Cetuximab, gemtuzumab ozogamicin and infliximab were also shown to contain N-glycolylneuraminic acid in their glycans, which can be prevented by using Neu5Gc-free media.

### Glycosites

In some embodiments, Fc regions and antibodies comprising Fc regions comprise a glycosite, e.g., an amino acid that can be glycosylated, whether or not the site is glycosylated. Generally, such sites comprise one or more atoms (e.g., nitrogen, oxygen, sulfur, carbon), optionally in one or more moieties (e.g., amino, amido, phenol, hydroxyl, guanidino, alcohol, thiol, indole), that are capable of forming a glycosidic bond with a sugar (e.g., glycosylation feature, such as a monosaccharide, oligosaccharide, polysaccharide, or derivative) molecule or part thereof. In some embodiments, a glycosite may comprise an amino acid comprising a side chain comprising an oxygen atom. In some embodiments, a glycosite may comprise an amino acid comprising a side chain comprising a sulfur atom. a glycosite may comprise an amino acid comprising a side chain comprising a nitrogen atom. The glycosite may comprise arginine, asparagine, serine, threonine, tyrosine, cysteine, homocysteine, ornithine, or lysine. In some embodiments, a glycosite may comprise a nucleic acid or portion (e.g., nucleotide) thereof. In some embodiments, a glycosite may comprise a lipid or portion thereof.

### Methods of Generating Antibodies

Fc regions and antibodies herein, in certain aspects, have modifications as compared to a wild-type Fc region or antibody. The modification may be one or more amino acid substitution (e.g., within 10 amino acids of a glycosite) as described elsewhere herein. Provided in this section are methods of preparing Fc regions and antibodies, where at least as used in this section, the antibodies may comprise Fc regions, or as applicable, may consist of Fc regions.

In various embodiments, antibodies are prepared using methods known in the art, such as, but not limited to the hybridoma method, where a host animal is immunized to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid by techniques known in the art, when propagated either in vitro or in vivo.

In some embodiments, antibodies are made using recombinant DNA methods. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, a chimeric antibody, a molecule in which different portions are derived from different animal species, such as those having a variable region derived from a murine monoclonal antibody and a human immunoglobulin constant region (e.g., humanized antibodies) can be generated.

In some embodiments, the antibody is a humanized antibody, to reduce antigenicity and HAMA (human anti-mouse antibody) responses when administered to a human subject. Humanized antibodies can be produced using various techniques known in the art. For example, an antibody is humanized by (1) determining the nucleotide and predicted amino acid sequence of the starting antibody light and heavy variable domains; (2) designing the humanized antibody, e.g., deciding which antibody framework region to use during the humanizing process; (3) the actual humanizing methodologies/techniques; and (4) the transfection and expression of the humanized antibody. In various embodiments, a humanized antibody can be further optimized to decrease potential immunogenicity, while maintaining functional activity, for therapy in humans.

Humanized antibodies can also be made in transgenic mice containing human immunoglobulin loci that are capable, upon immunization, of producing the full repertoire of human antibodies in the absence of endogenous immunoglobulin production. A humanized antibody may also be obtained by a genetic engineering approach that enables production of affinity-matured human-like polyclonal antibodies in large animals.

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications should therefore be restricted to a minimum and closely watched for changes in the specificity and affinity of the antibody. On the other hand, framework residues in theory can be derived from any human variable region. A human framework sequences should be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g., mutated or affinity matured) sequences. Genetic engineering techniques well known to those in the art, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR.

In certain embodiments, the antibody is a human antibody. Human antibodies can be directly prepared using various techniques known in the art. Immortalized human B lymphocytes immunized in vitro or isolated from an immunized individual that produce an antibody directed against a target antigen can be generated.

Chimeric, humanized and human antibodies may be produced by recombinant expression. Recombinant polynucleotide constructs typically include an expression control sequence operably linked to the coding sequences of antibody chains, including naturally associated or heterologous promoter regions. In certain embodiments, it may be desirable to generate amino acid sequence variants of these humanized antibodies, particularly where these improve the binding affinity or other biological properties of the antibody.

In various embodiments, the expression of an antibody can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody may be transfected into the host.

In some embodiments, the expression vectors are transfected into the recipient cell line for the production of the antibodies. In various embodiments, mammalian cells can be useful as hosts for the production of antibody proteins, which can include, but are not limited to cells of fibroblast origin, such as Vero (ATCC CRL 81) or CHO-K1 (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO—S and DG44 cells; PER.C6™ cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying an antibody construct can be introduced into an appropriate host cell by any of a variety of suitable means, depending on the type of cellular host including, but not limited to transformation, transfection, lipofection, conjugation, electroporation, direct microinjection, and microprojectile bombardment, as known to one of ordinary skill in the art. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody. In various other embodiments, bacterial strains can also be utilized as hosts for the production of the antibody. Examples of bacterial strains include, but are not limited to E. coli, Bacillus species, enterobacteria, and various Pseudomonas species.

In some embodiments, antibodies can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like.

Once expressed in the host, the whole antibodies can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. Once purified, partially or to homogeneity as desired, an antibody can then be used therapeutically.

Various embodiments provide for a genetic construct comprising a nucleic acid encoding an antibody or fragment provided herein. Genetic constructs of the antibody can be in the form of expression cassettes, which can be suitable for expression of the encoded antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by methods known in the art. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3T3, Chinese hamster ovary (CHO), HeLa and BHK cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

In some embodiments, the antibody or fragment thereof is a variant of another antibody or fragment thereof. Alterations of the native amino acid sequence can be accomplished by any of a number of techniques known to one of skill in the art. Mutations can be introduced at particular loci or by oligonucleotide-directed site-specific mutagenesis procedures.

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, NY, 1982 and 1989), and can be used to construct nucleic acid sequences which encode a monoclonal antibody molecule or antigen-binding region.

### Pharmaceutical Compositions and Methods of Treatment

Also described herein are pharmaceutical compositions, wherein a pharmaceutical composition may comprise a Fc region or antibody comprising a Fc region as described herein or a fragment thereof. In some embodiments, a pharmaceutical composition may further comprise a pharmaceutically acceptable carrier, an excipient, or any combination thereof. A “pharmaceutically acceptable carrier or excipient” may comprise one or more molecular entities that do not materially affect the composition or change the active agent(s) contained therein, are physiologically tolerable, and do not typically produce an allergic reaction, or similar untoward reaction, when administered to a subject.

Also described herein are methods for treating a subject using a formulation or pharmaceutical composition as described herein. Also described herein are methods for prophylactic treatment of a subject using a formulation or pharmaceutical composition as described herein. Pharmaceutical compositions are formulated in a conventional manner using one or more pharmaceutically acceptable excipients that facilitate processing of the active compounds, i.e., modified glycoproteins or functional fragments thereof, into preparations that may be used pharmaceutically. Proper formulation is dependent upon the route of administration chosen. A summary of pharmaceutical compositions described herein may be found, for example, in Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Such methods may comprise administering to a subject an effective amount of the pharmaceutical composition or formulation. An effective amount may be determined, for example, based on the KD of a modified glycoprotein within the formulation or pharmaceutical composition, the bioavailability of a modified glycoprotein within the formulation or pharmaceutical composition, the route of administration of the formulation or pharmaceutical composition, other factors, or a combination thereof.

In some embodiments, a formulation or pharmaceutical composition may further comprise a second therapeutic. For example, a formulation or pharmaceutical composition may further comprise a pain reliever (e.g., ibuprofen or acetaminophen or any other suitable pain reliever), an antiviral compound (e.g., remdesivir or any other suitable antiviral compound), an antibiotic compound (e.g., azithromycin or any other suitable antibiotic compounds) or a steroid (e.g., dexamethasone, corticosteroids, cortisone, hydrocortisone, prednisone, or any other suitable steroids).

In some embodiments, a method may further comprise administering a pain reliever (e.g., ibuprofen or acetaminophen), an antiviral compound (e.g., remdesivir), an antibiotic compound (e.g., asithromycin) or a steroid (e.g., dexamethasone). In some embodiments, the second therapeutic compositions may be administered prior to the administration of the modified glycopeptides or the functional fragments thereof disclosed therein. In some embodiments, the second therapeutic compositions may be administered subsequent to the administration of the modified glycoproteins or the functional fragments thereof disclosed therein. In some embodiments, the second therapeutic compositions may be administered at the same time to the administration of the modified glycopeptides or the functional fragments thereof disclosed therein. In some embodiments, the second therapeutic may be conjugated to the modified glycopeptide.

### EXAMPLES

### Example 1. Modulation of ADCC

Antibodies are engineered with one or more amino acid substitutions in the Fc region to generate mutant Fc antibodies (mutant Fc Herceptin, mutant Fc Rituximab). The accuracy and generalizability of predictions for antibody Fc substitutions is determined. The stability of the mutant antibodies is determined. Table 1 provides a list of mutations. The numbering is EU numbering.

TABLE 3
Fc Mutations

| Index (EU) | core-fucose |
|---|---|
| P291E | − |
| P291I | |
| P291K | − |
| P291L | |
| P291Q | − |
| P291R | N.C. (no change) |
| P291V | |
| R292E | − |
| R292K | − |
| R292Q | − |
| Q295E | − |
| Q295K | + |
| Q295R | + |
| Y296E | − |
| Y296F | + |
| Y296I | |
| Y296K | − |
| Y296Q | − |
| Y296R | N.C. |
| Y296V | |
| Y296W | N.C. |
| S298F | |
| S298H | |
| S298N | |
| S298T | |
| S298W | |
| S298Y | |
| Y300F | + |
| Y300F | |
| Y300H | |
| Y300I | |
| Y300I | |
| Y300L | |
| Y300V | |
| Y300V | |
| Y300W | |
| Y300W | |
| R301E | − |
| R301H | |
| R301K | − |
| R301Q | − |
| R301W | |
| V302F | |
| V302H | |
| V302I | |
| V302L | |
| V302Q | |
| V302W | |
| V302Y | |
| V303F | |
| V303H | |
| V303I | |
| V303L | |
| V303Q | |
| V303W | |
| V303Y | |
| S304F | |
| S304H | |
| S304N | |
| S304T | |
| S304W | |
| S304Y | |
| E294H | |
| E294K | |
| E294Q | |
| E294R | |
| E294W | |
| E293H | |
| E293K | |
| E293Q | |
| E293R | |
| E293W | |
| P291I; V303F | − |
| V302F; V303F | − |
| P291Q; V303F | − |
| P291L; V303F | − |
| P291L; S304N | − |
| V303F; S304N | − |
| V303F; S304T | − |
| V303F; S304F | − |
| P291L; V303W | − |
| P291Q; S304N | − |
| P291I; V303W | − |
| V302W; V303F | − |
| V302W; V303W | − |
| V302F; V303W | − |
| P291I; V302F | − |
| P291Q; S304F | − |
| P291Q; R292Q | − |
| P291L; S304F | − |
| P291Q; S304T | − |
| P291L; V303Q | − |
| V302H; V303F | − |
| R292Q; V303F | − |
| P291Q; V302F | − |
| P291L; V302F | − |
| V302Q; V303F | − |
| P291I; V303Q | − |
| V302Y; V303W | − |
| P291I; S304N | − |
| V302F; S304N | − |
| V302Q; V303W | − |
| K290L; P291L | − |
| P291Q; T207N | − |
| K290L; V303Q | − |
| K290L; V303W | − |
| K290I; P291L | − |
| R292Q; T207N | − |
| P291E; T207N | − |
| K290L; V303F | − |
| K290L; V303Y | − |
| K290L; V303I | − |
| K290I; V303F | − |
| K290I; V303Q | − |
| P291L; T207N | − |
| K290L; P291Q | − |
| K290E; V303F | − |
| K290E; R292Q | − |
| K290L; V302Q | − |
| K290L; R292Q | − |
| K290L; V303H | − |
| K290L; V302W | − |
| K290E; V303Q | − |
| K290E; V303Y | − |
| K290L; V302I | − |
| K290L; V302Y | − |
| K290E; V303H | − |
| K290E; V302Q | − |
| K290L; V302F | − |
| K290I; V302Q | − |
| K290I; V303W | − |
| K290E; P291L | − |

(+ is an expected increase in fucose, − is an expected decrease)

TABLE 4
Modified antibodies (each mutant, e.g., mut1, mut2, etc.
will comprise at least one substitution from Table 3)

| Sample/ | | Genotype/ | | structure | Glyco- | | |
|---|---|---|---|---|---|---|---|
| Flask | Ab | Plasmid | Replicate | impact | impact | Proximity * | type |
| 1 | Rituximab | wt | 1 | | | | |
| 2 | Rituximab | mut1 | 1 | low | low | far | neg control |
| 3 | Rituximab | mut2 | 1 | low | high | far | neg control |
| 4 | Rituximab | mut3 | 1 | low | low | close | neg control |
| 5 | Rituximab | mut4 | 1 | low | high | close | experimental |
| 6 | Rituximab | mut5 | 1 | low | high | close | experimental |
| 7 | Rituximab | mut6 | 1 | low | | | |
| 8 | Rituximab | mut7 | 1 | low | | | |
| 9 | Rituximab | mut4 | 2 | low | high | close | experimental |
| 10 | Rituximab | mut5 | 2 | low | high | close | experimental |
| 11 | Herceptin | wt | 1 | low | | | |
| 12 | Herceptin | mut1 | 1 | low | | | |
| 13 | Herceptin | mut2 | 1 | low | | | |
| 14 | Herceptin | mut3 | 1 | low | | | |
| 15 | Herceptin | mut4 | 1 | low | | | |
| 16 | Herceptin | mut5 | 1 | low | | | |
| 17 | Herceptin | mut6 | 1 | low | | | |
| 18 | Herceptin | mut7 | 1 | low | | | |
| 19 | Herceptin | mut1 | 2 | low | | | |
| 20 | Herceptin | mut2 | 2 | low | | | |
| 21 | Rituximab | wt + FUT8 inhibitor | 1 | | | | |
| 21 | Rituximab | wt + a16 fucosidase | 1 | | | | |

* near/far mutants should be matched; same mutant near and far

Proximity indicates how close in three-dimensional space the mutation is to the fucosylation site

TABLE 5
Methods and Approach

| | | | Est. | |
|---|---|---|---|---|
| # | Steps | Method/Origin | Duration | |
| | Work Package 1A | | | |
| 1 | Design synthetic gene of | A synthetic gene of Rituximab will be | 1 | week |
| | Rituximab | designed, with unique silent restriction | | |
| | | sites around the N-glycosylation site in | | |
| | | the DNA sequence in the expression | | |
| | | plasmid, and introducing the recognition | | |
| | | sites of enzymes BaeI/XhoI/HpaI. The | | |
| | | gene will be inserted into an expression | | |
| | | vector chosen by DTU. | | |
| 2 | Procurement of a synthetic | Purchase from vendor | 3 | weeks |
| | gene of Rituximab | | | |
| 3 | Design the 95 mutant variants | Using Augment Biologics' confidential | | |
| | to be introduced (single and | approach, glycan-modulating mutations | | |
| | double mutations are | will be selected, prioritized and | | |
| | possible) | communicated to DTU. | | |
| 4 | Generation of up to 95 | Establish expression vector using specific | 6 | weeks |
| | variants of Rituximab, with | oligonucleotides and restriction enzymes | | |
| | single or double mutations | to insert mutated sequences encoding the | | |
| | around the glycosylation site | region around the glycosylation site. | | |
| | N297 in the heavy chain, | The specific oligo's mutations will be | | |
| | numbering as in Exhibit 2. | introduced in the mutational window of −4 | | |
| | | to +8 PREEQYNSTYRVVSVLT (SEQ | | |
| | | ID NO: 2) and the entire region between | | |
| | | XhoI and HpaI will be replaced (−4 to +8). | | |
| 5 | Selection of 30 variants from | Select 30 variants to be expressed by | 1 | week |
| | the variants generated above | transient transfection in CHO cells. | | |
| | for transient expression | | | |
| 5 | Transient transfection of | Prepare DNA for transient transfection, | 3 | weeks |
| | Rituximab (anti-CD20 IgG1) | thaw and expand CHO cells. Perform 5 | | |
| | and up to 30 Rituximab | days transient expression in 30 ml shake | | |
| | variants + in 30 ml tissue | flasks. | | |
| | culture flasks. | | | |
| 5 | Titer Determination of | Determine Rituximab titer in the above | 2 | d |
| | Rituximab in the above 31 | samples, using protein A based SPR | | |
| | samples | technique on a RED96 Octet. | | |
| | | Measurements will be run in | | |
| | | duplicate/triplicate. | | |
| 6 | Determine presence of | SDS-PAGE and Western blotting using a | 2 | d |
| | Rituximab in the above 31 | Rituximab-specific antibody | | |
| | samples | | | |
| 7 | Storage of harvested | Harvest supernatants, sample 3X1 ml and | | |
| | supernatants | store remaining supernatant at −80 C. | | |
| | | Each aliquot will be sampled for titer and | | |
| | | identity prior to storage. | | |
| | | The remaining supernatants will be stored | | |
| | | at −80 C. These will then be thawed for | | |
| | | purification when needed ensuring a | | |
| | | maximum of one freeze thaw cycle for | | |
| | | any aliquot. | | |
| 8 | Write comprehensive report, | Send report and pdf of ELN to The | | |
| | finalize electronic lab | Company | | |
| | notebook (ELN) | | | |
| 9 | Raw Data | All raw and analyzed data as well as | | |
| | | Benchling ELN entries are stored on | | |
| | | backed up servers at DTU. | | |
| | Work Package 1B | | | |
| 10 | Data analysis | The Company will select 5 variants to be | | |
| | | purified from the transient transfection | | |
| 11 | 1-step Purification of 5 + 1 | Purification of 5 selected variants from | 3 | d |
| | selected variants from | above (selected by Company) + | | |
| | supernatants generated in | Rituximab on ÄKTA Pure equipment | | |
| | work package 1a | using MabSelect columns | | |
| | | (https://www.cytivalifesciences.com/en/us/about- | | |
| | | us/our-brands/mabselect) | | |
| 12 | Storage of purified protein | Aliquots of the purified proteins will be | | |
| | | stored at −80 C. in liquid buffer. | | |
| | | The number and volume of the aliquots | | |
| | | depends on the yield targeting a | | |
| | | concentration of 1 mg/mL and | | |
| | | 0.5 ml/aliquot. | | |
| 13 | QC Purity & Yield | SDS-PAGE and A280 respectively | 2 | d |
| 14 | Write a comprehensive report, | Send report and pdf of ELN to The | | |
| | finalize ELN | Company | | |
| 15 | Raw data | All raw and analyzed data as well as | | |
| | | Benchling ELN entries are stored on | | |
| | | backed up servers at DTU. | | |
| | Work Package 2 | | | |
| 16 | Enzymatic defucosylation of | Treatment of purified Rituximab variants | 3 | d |
| | purified proteins from above | from above with fucose-exoglycosidase | | |
| | Samples: | (New England Biolabs: P0748S) | | |
| | standard Rituximab and 5 | according to the protocol. | | |
| | Rituximab variants (output of | (https://international.neb.com/protocols/2015/02/26/typical- | | |
| | task #10) | reaction-conditions-for- | | |
| | | 1-2-4-6-fucosidase-p0748) | | |
| 17 | MS based glycoprofiling of | N-Glycan analysis by online liquid | 1 | w |
| | 12 samples (non- | chromatography-MS (LC-MS) using an | | |
| | defucosylated and fucosylated | Orbitrap Fusion Tribrid mass | | |
| | from above) | spectrometer (Thermo Fisher Scientific) | | |
| | | coupled to a Thermo Ultimate 3000 | | |
| | | HPLC system (Thermo Fisher Scientific). | | |
| | | Released N-glycans from glyco-proteins | | |
| | | of interest are fluorescently labeled with | | |
| | | GlycoWorks RapiFluor- MS N-Glycan | | |
| | | Kit (Waters, Milford, MA) as per the | | |
| | | manufacturer's protocol. | | |
| 18 | Write a comprehensive report, | Send report and pdf of ELN to The | | |
| | finalize ELN | Company | | |
| 19 | Raw data | All raw and analyzed data as well as | | |
| | | Benchling ELN entries are stored on | | |
| | | backed up servers at DTU. | | |
| | Work Package 3a | | | |
| 20 | Antibody-dependent cellular | Promega ADCC Reporter Bioassay, V | 1 | week |
| | cytotoxicity (ADCC) assay | Variant according to protocol | | |
| | 12 samples (non- | | | |
| | defucosylated and de- | | | |
| | fucosylated as described in | | | |
| | workpackage 2) | | | |
| | Total samples: 12 | | | |
| 21 | Write a comprehensive report, | Send report and pdf of ELN to The | | |
| | finalize ELN | Company | | |
| 22 | Raw data | All raw and analyzed data as well as | | |
| | | Benchling ELN entries are stored on | | |
| | | backed up servers at DTU. | | |
| | Work package 3b | | | |
| 23 | Kn/Kd measurements of the | Octet/K_{D} (kinetic assay using biotinylated | 1 | week |
| | 12 proteins affinity to CD20. | CD20 as bound target on the SPR tips. | | |
| | testing affinity to e.g. His- | | | |
| | tagged/biotinylated CD20 | | | |
| | Total samples: 12 | | | |
| 24 | Write a comprehensive report, | Send report and pdf of ELN to The | | |
| | finalize ELN | Company | | |
| 25 | Raw data | All raw and analyzed data as well as | | |
| | | Benchling ELN entries are stored on | | |
| | | backed up servers at DTU. | | |
| | Completion/Delivery/Post- | | | |
| | term | | | |
| 26 | Materials | Materials refer to Results (defined in | | |
| | disposal//return/storage | section 1) to include any designs, | | |
| | If returned, Company will pay | electronic records, notes, records, notes, | | |
| | for shipping, etc | biological materials, biological | | |
| | | derivatives, and anything of material | | |
| | | importance during produced within the | | |
| | | execution of the Tasks or over the course | | |
| | | of the project. | | |
| 27 | Maintenance of materials | Fees will include 5 years of storage at the | | |
| | | DTU facility. After 5 years. On the | | |
| | | Company's written request, storage can be | | |
| | | extended beyond 5 years at a rate of | | |
| | | $500/month. All materials will be | | |
| | | destroyed at the Company's written | | |
| | | request or at the end of the agreed upon | | |
| | | storage term. | | |
| 28 | Use of materials | Materials cannot be used for any purpose | | |
| | | other than those delineated in the forgoing | | |
| | | contract without written approval from the | | |
| | | Company | | |
| 29 | Return and disposal of | Legally transferable materials (to exclude | | |
| | materials | non-transferable materials including | | |
| | | expiCHO cell lines and constructed | | |
| | | vectors) will be transferred promptly (no | | |
| | | more than 30 days) upon request. All | | |
| | | materials will be destroyed promptly upon | | |
| | | the Company's written request. Any | | |
| | | transfer fees (including but not limited to | | |
| | | shipping, handling and duties) will be paid | | |
| | | by the Company. | | |

Substitutions modify fucose glycosylation of the antibodies, thereby modulating antibody ADCC, CDC, and/or ADCP.

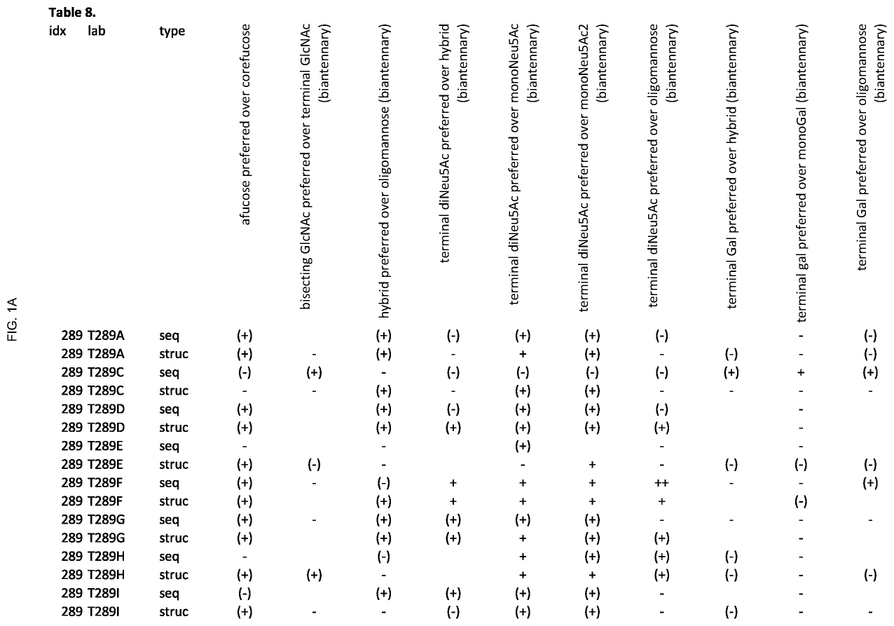

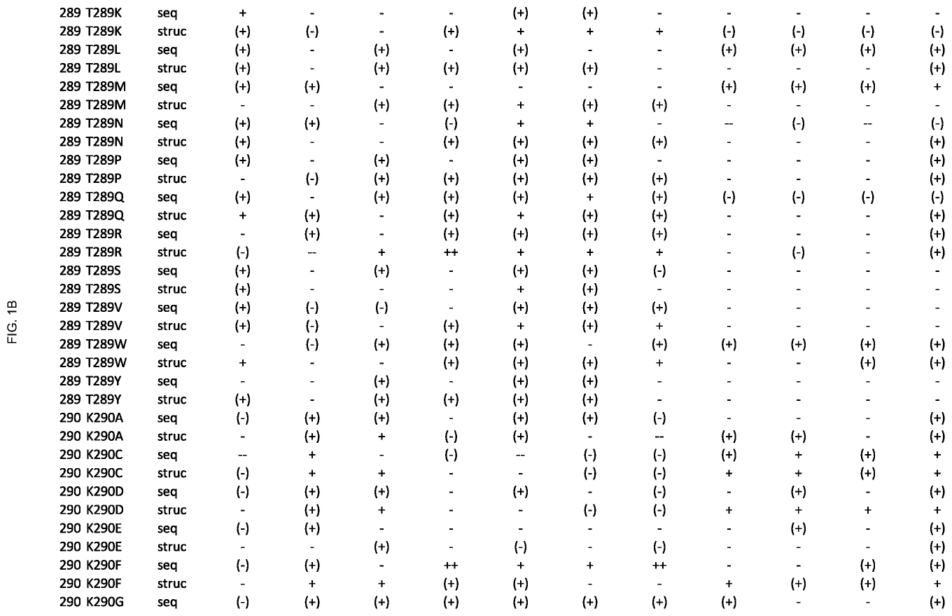

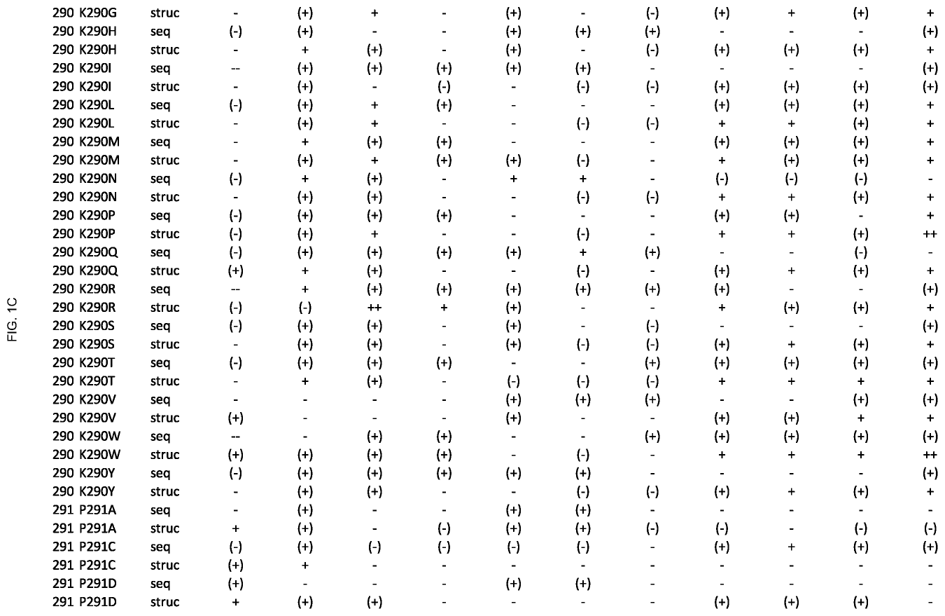

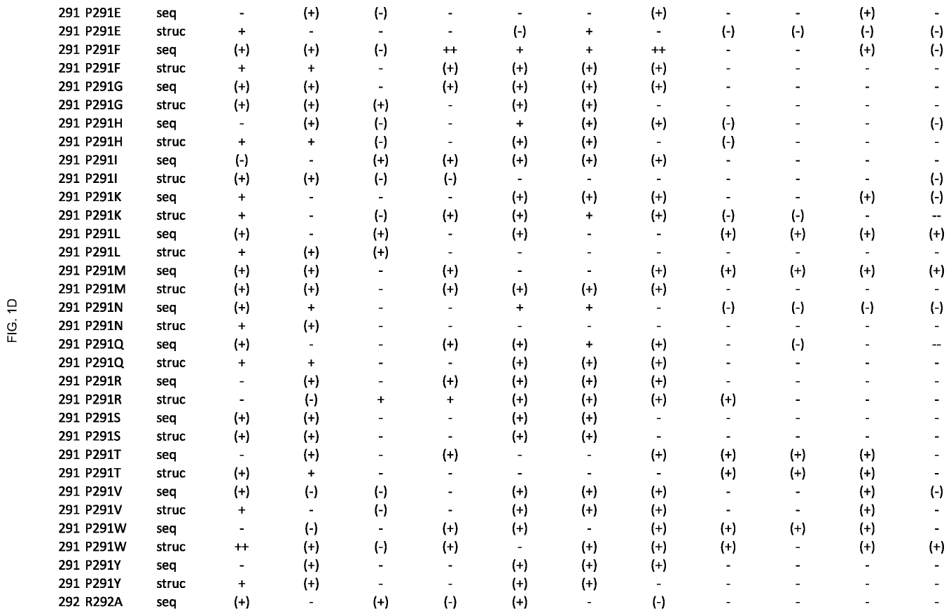

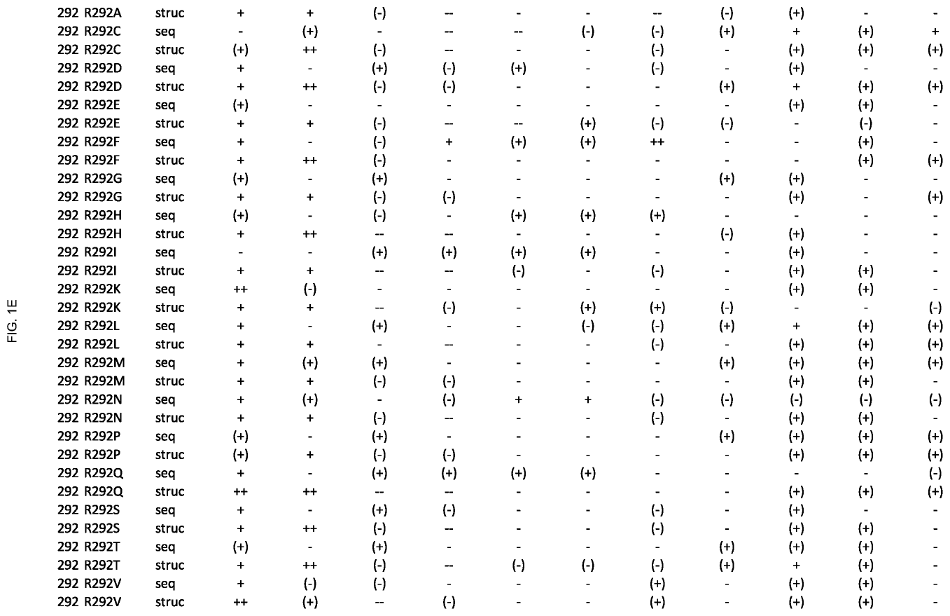

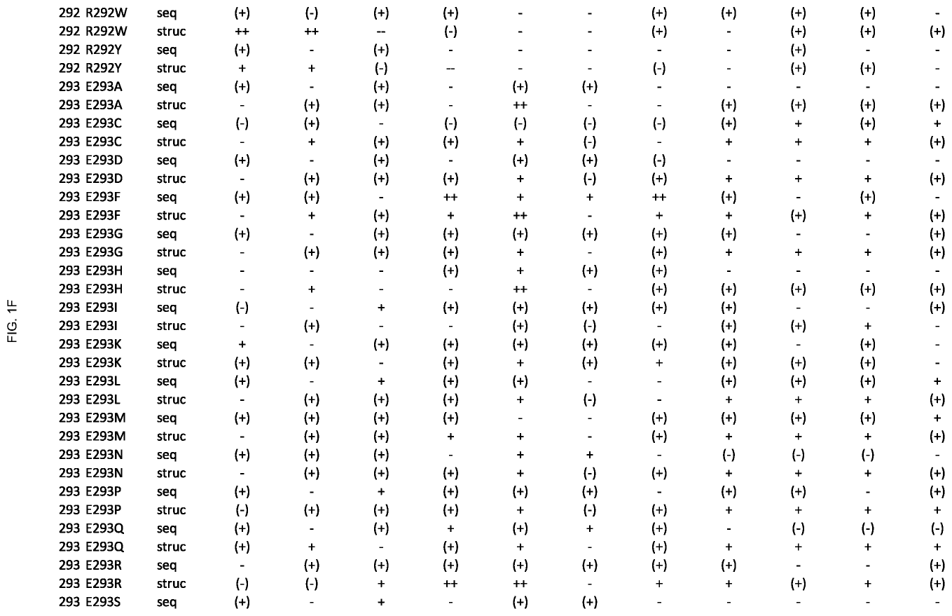

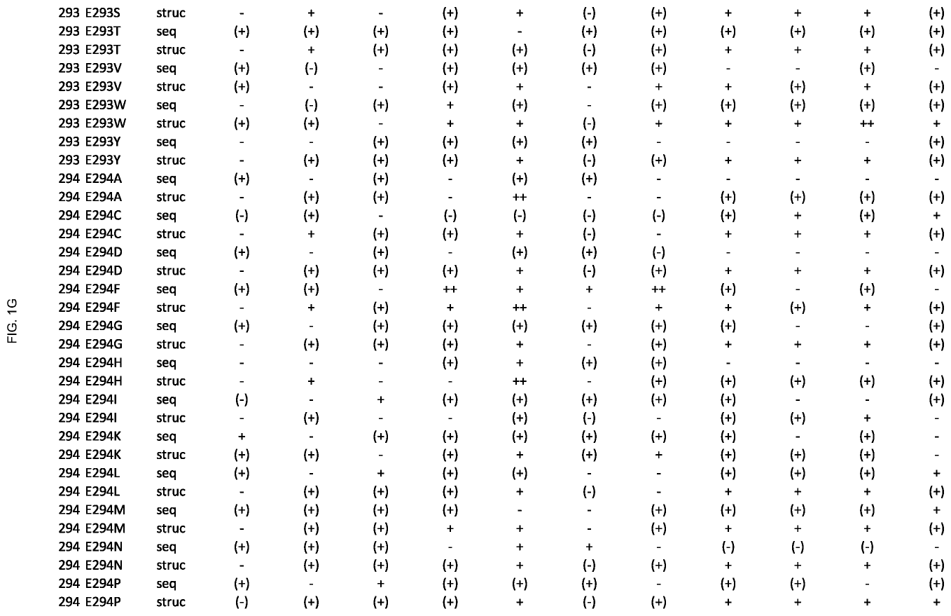

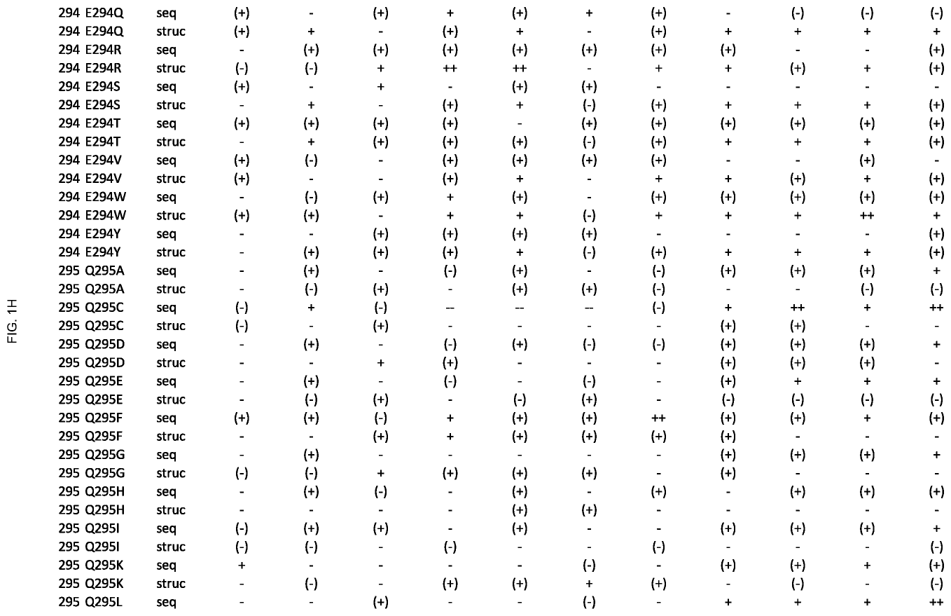

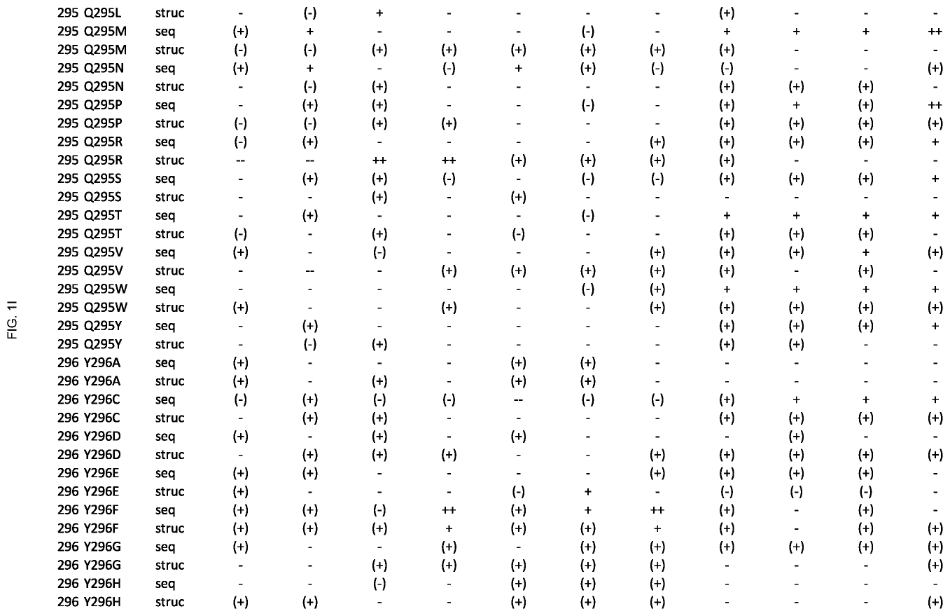

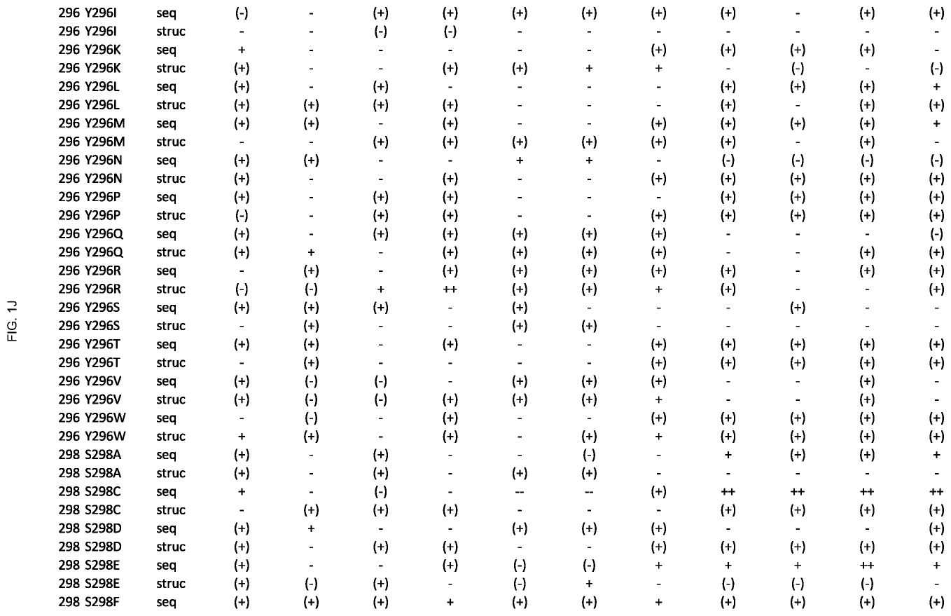

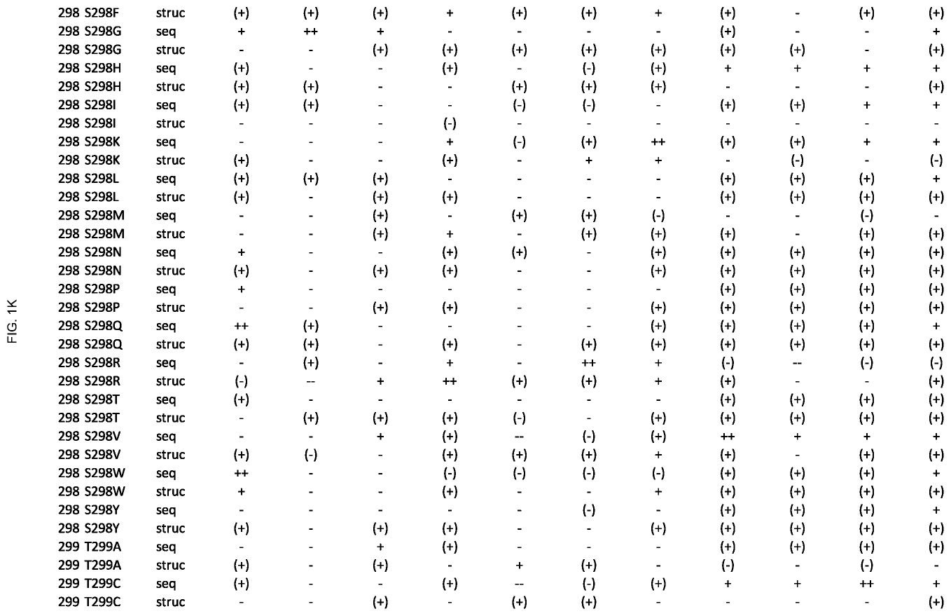

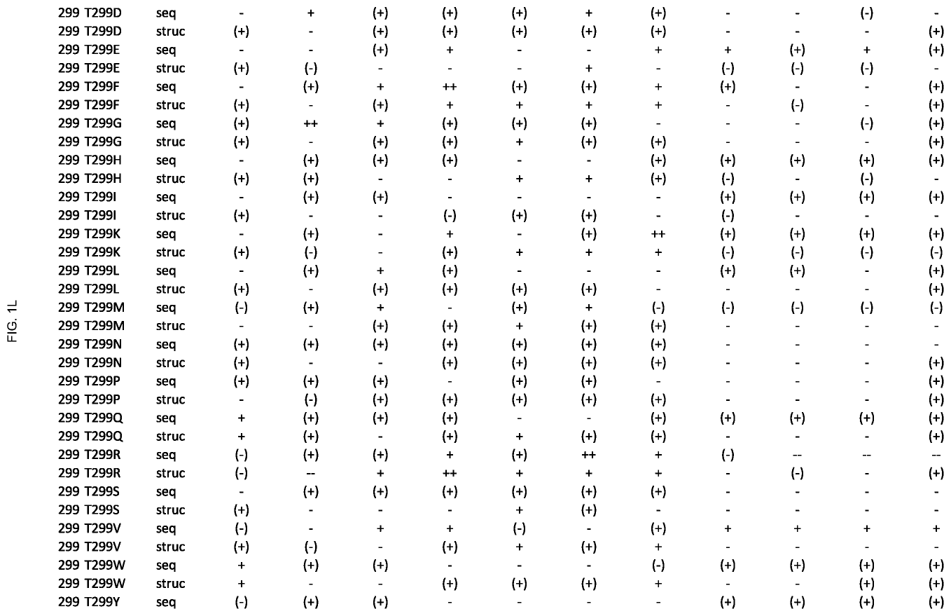

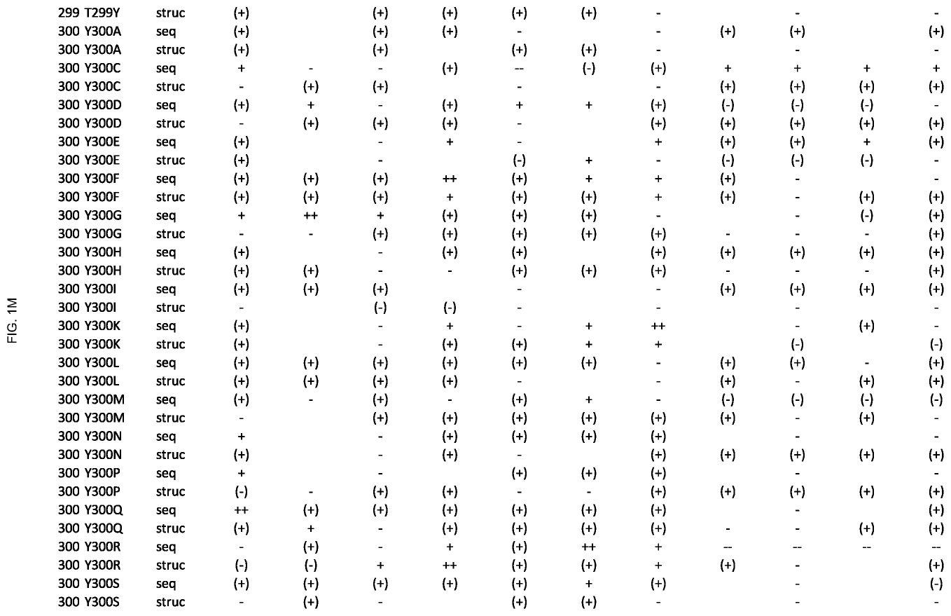

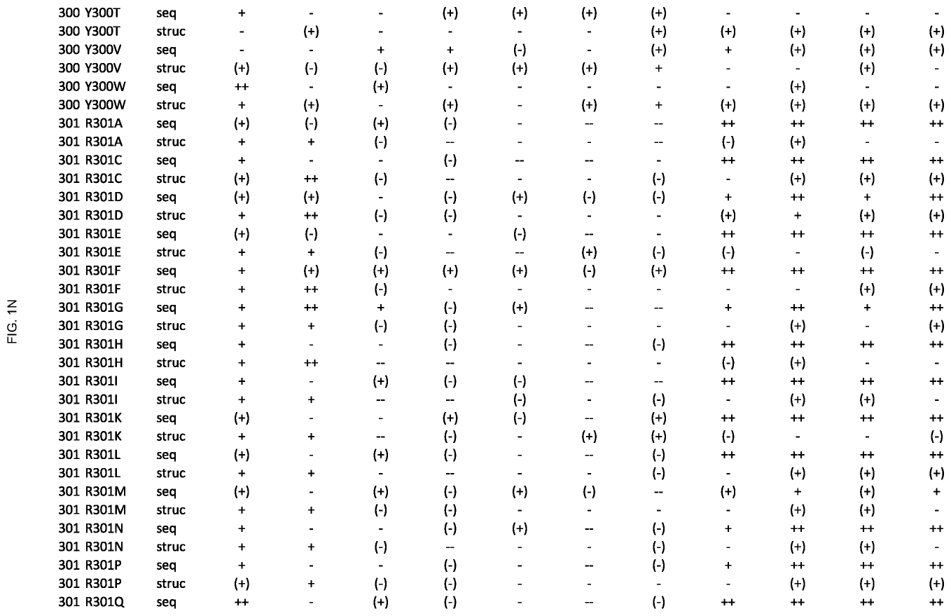

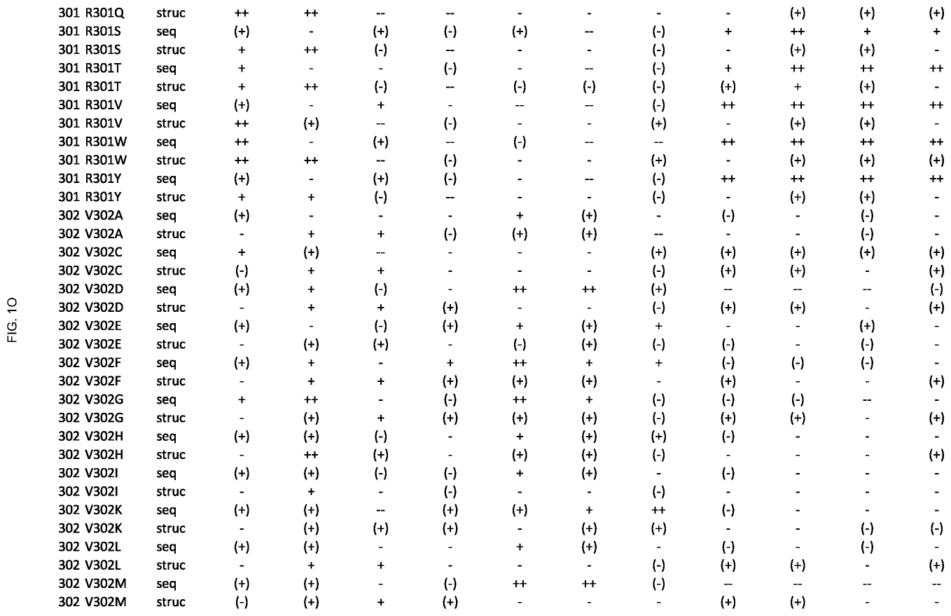

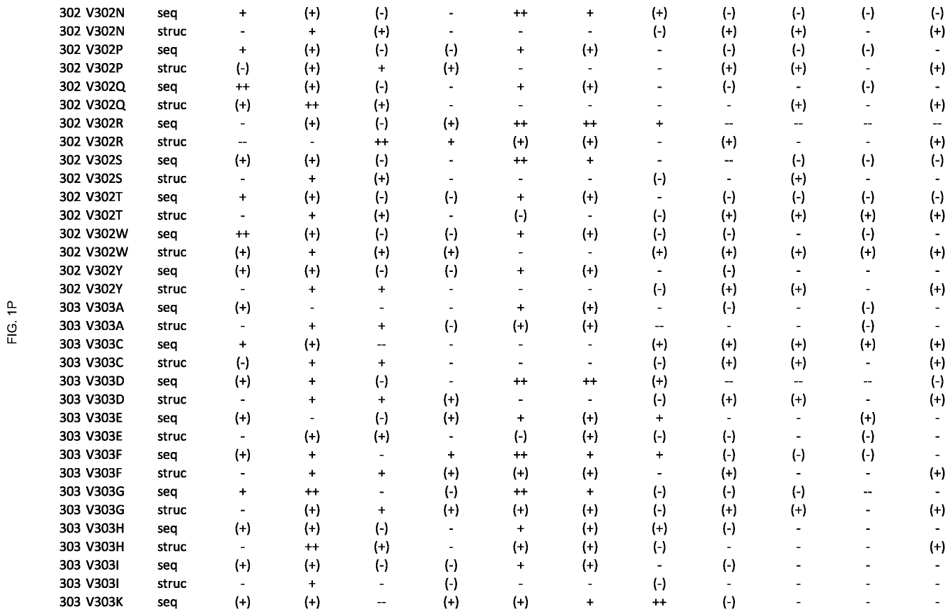

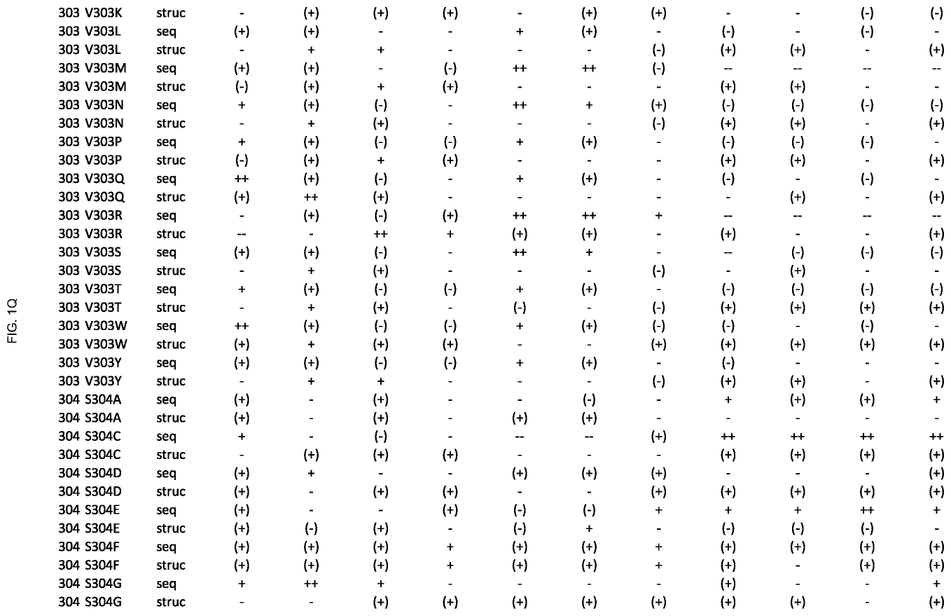

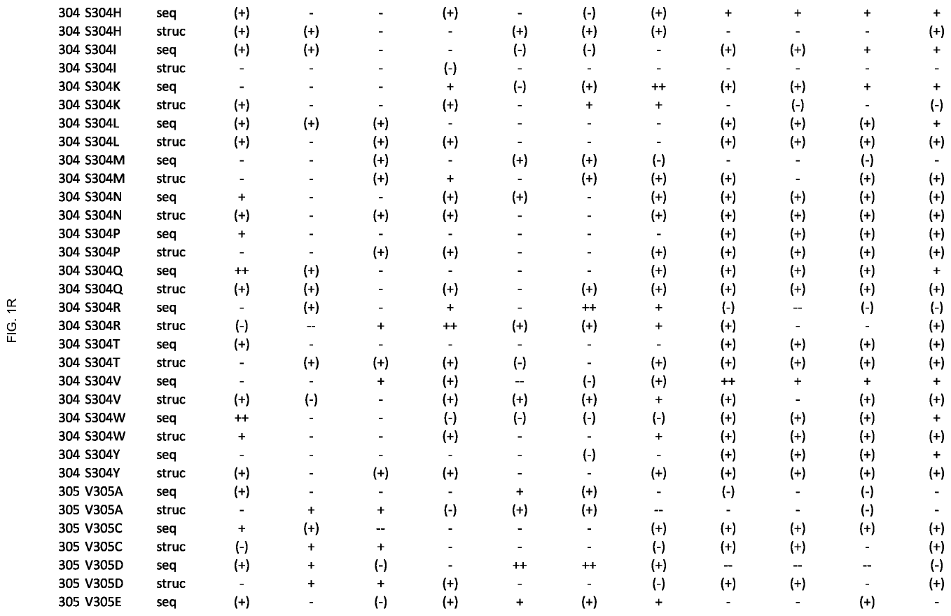

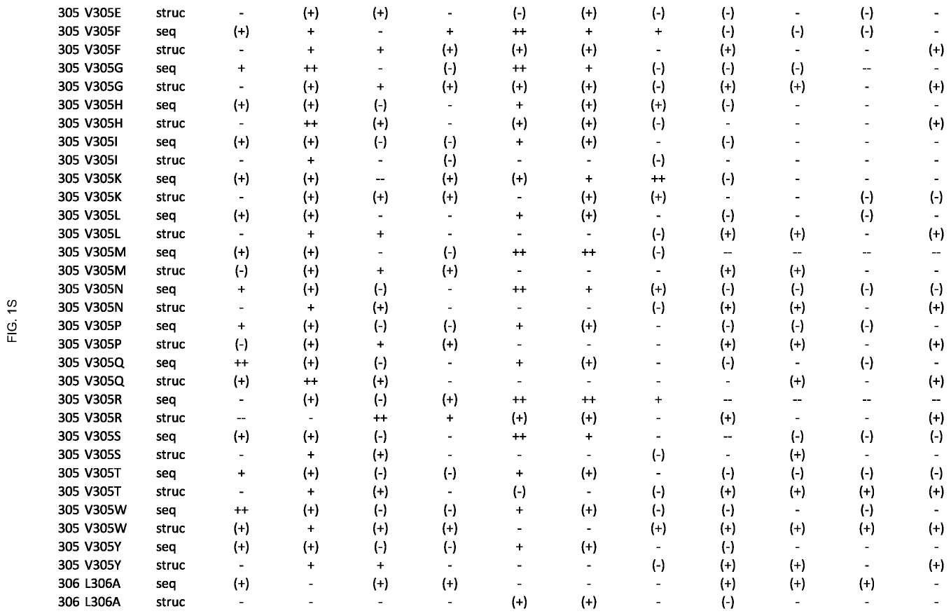

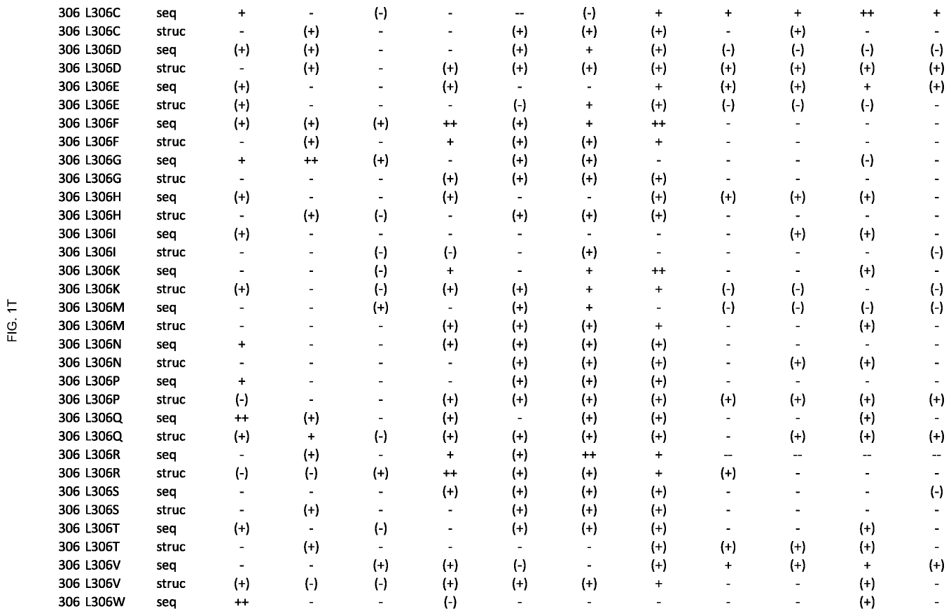

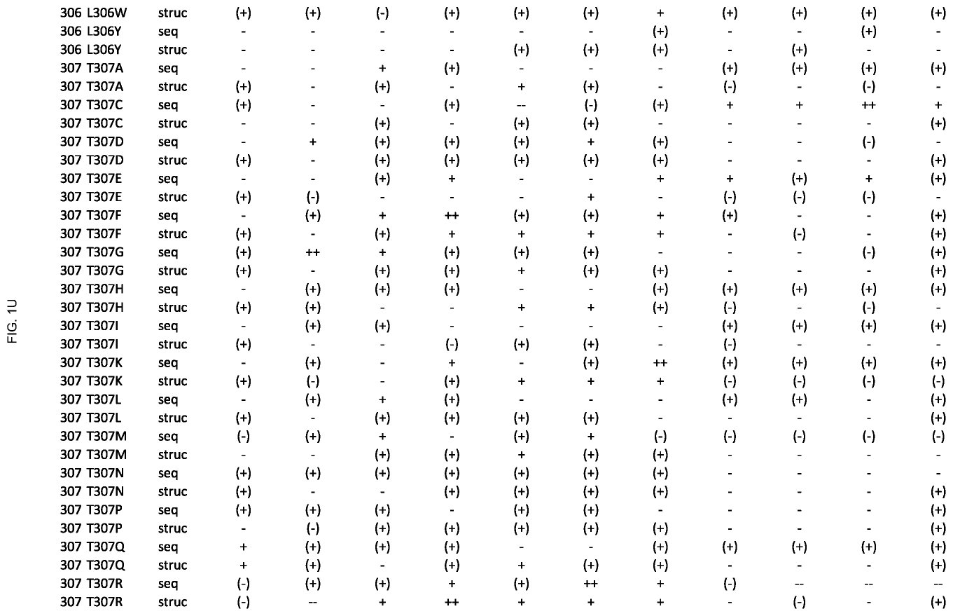

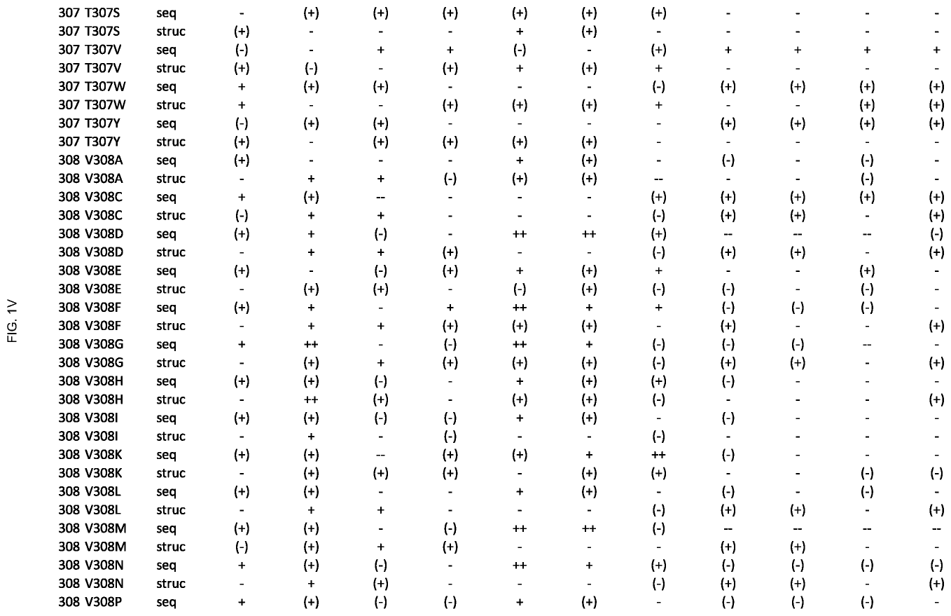

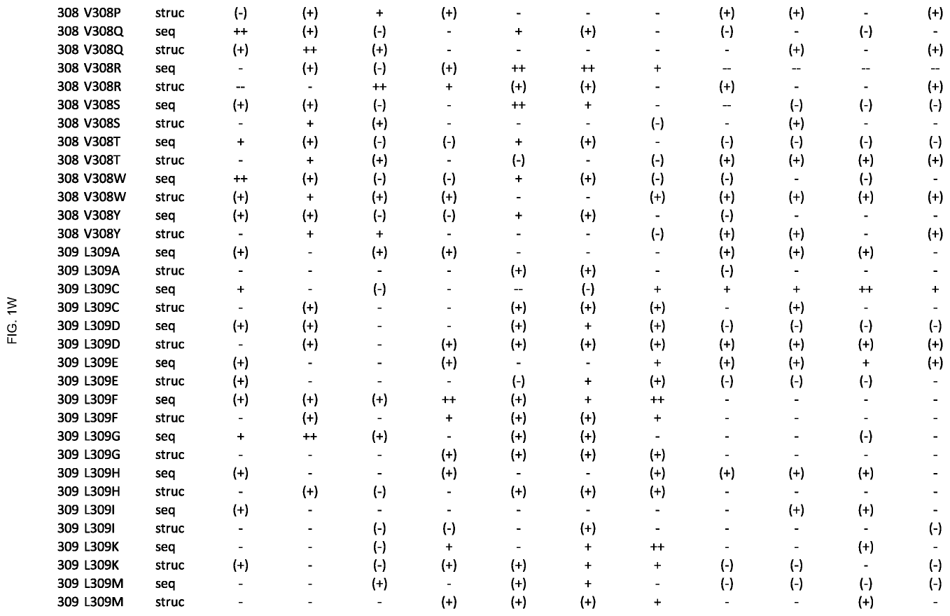

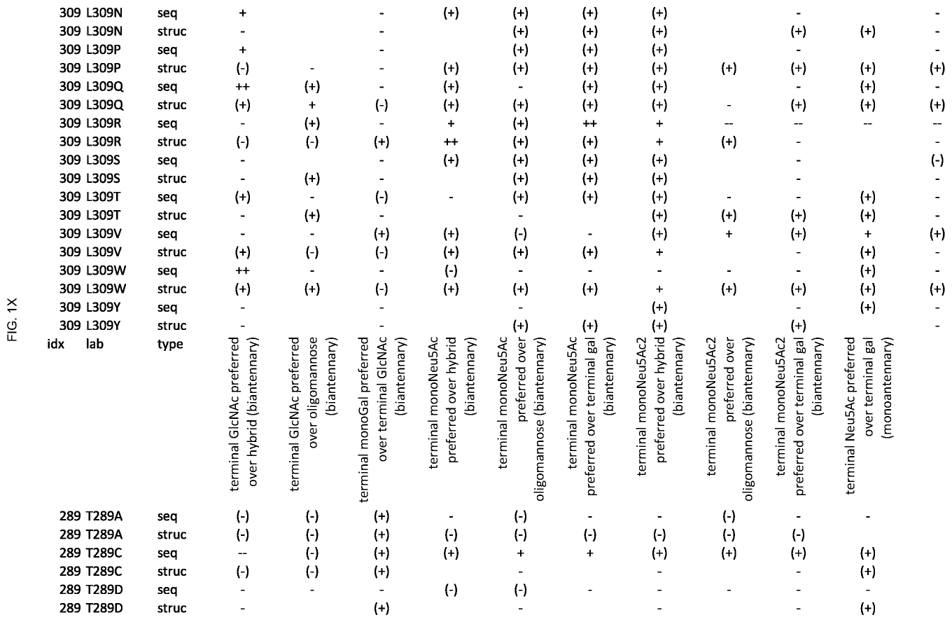

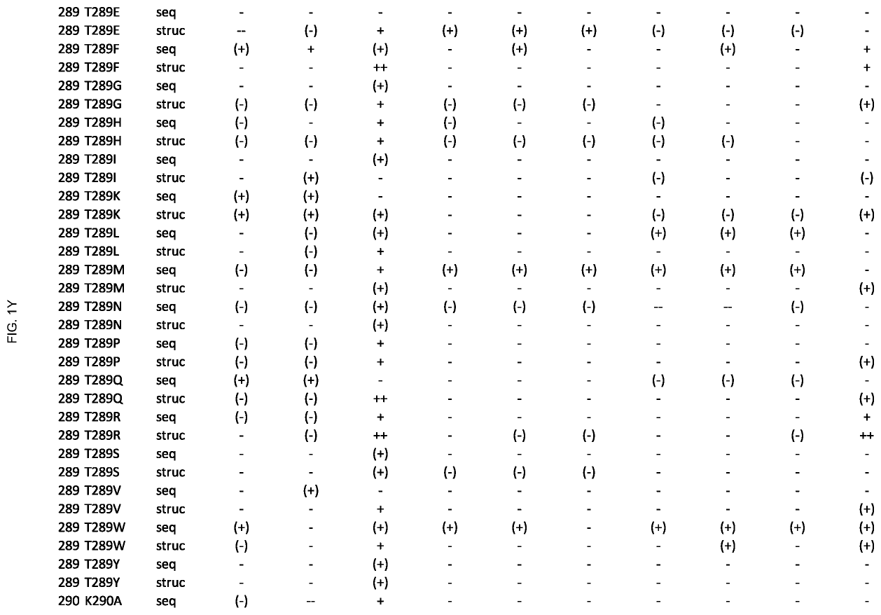

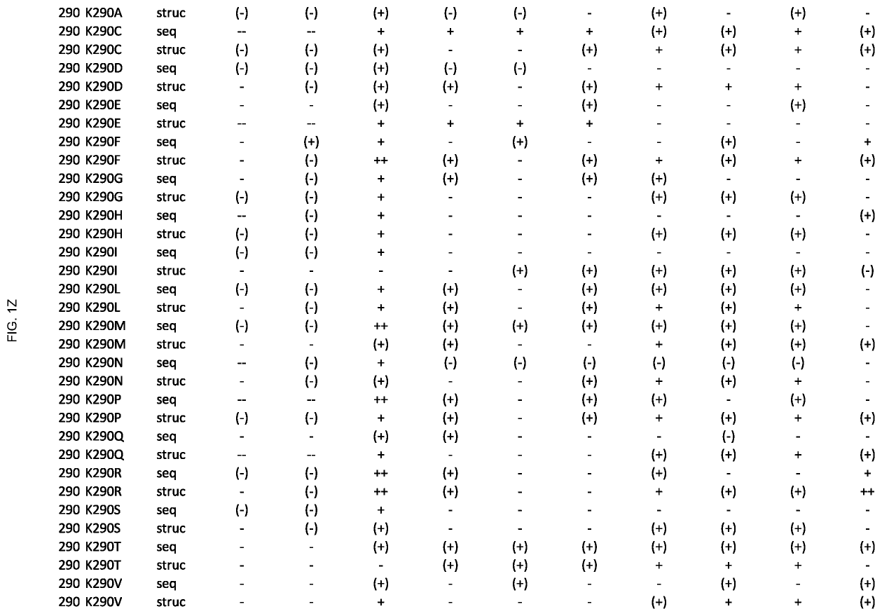

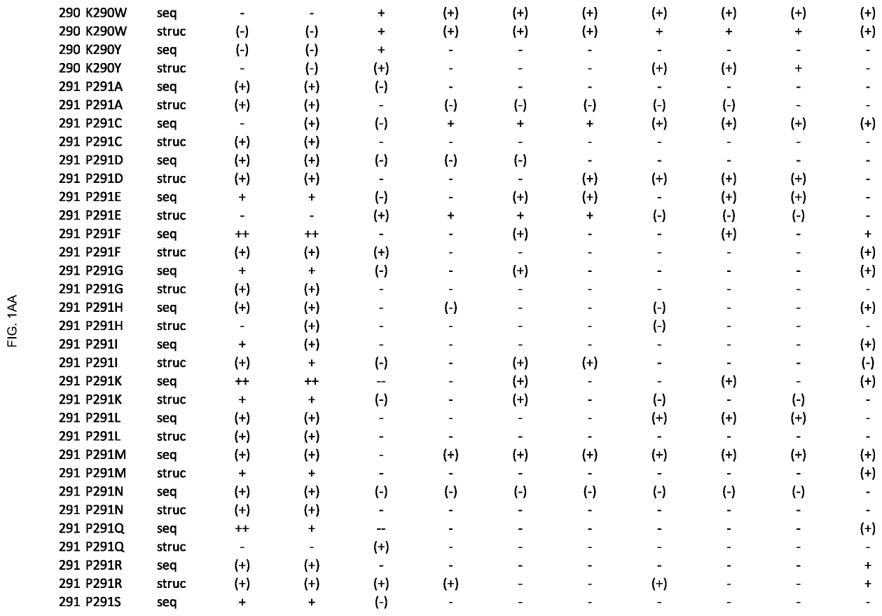

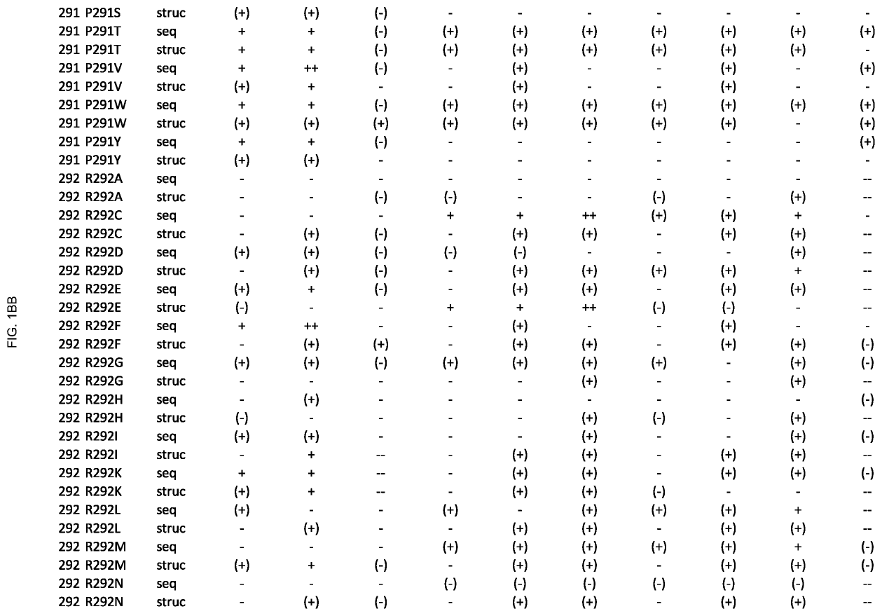

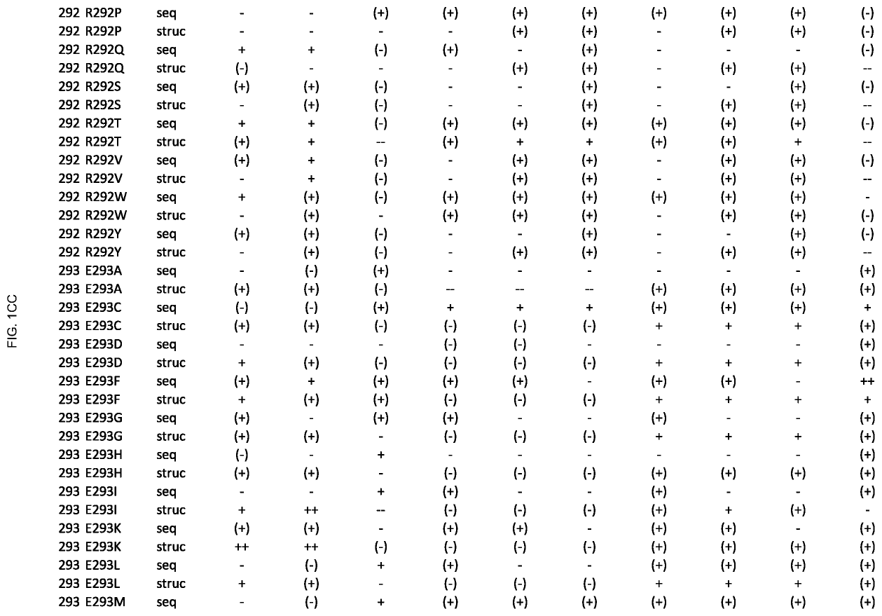

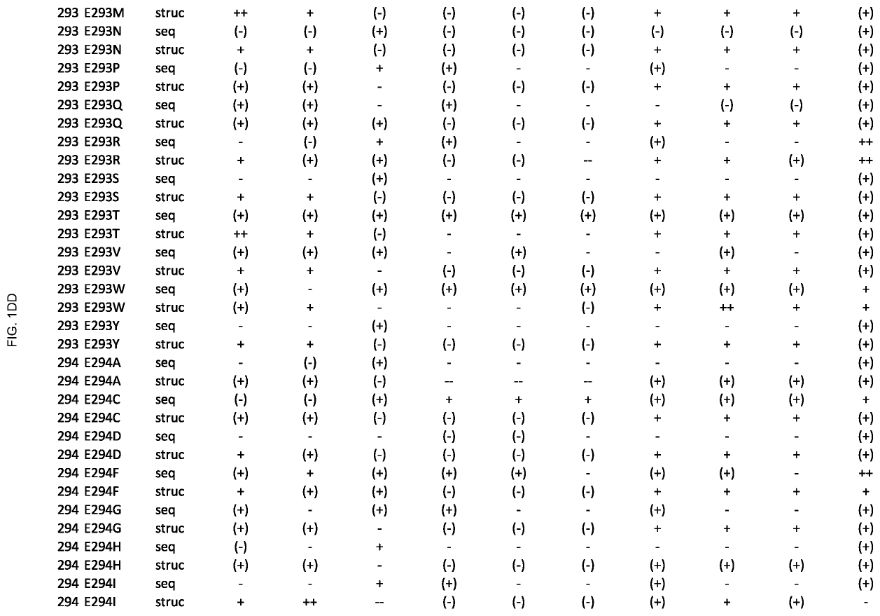

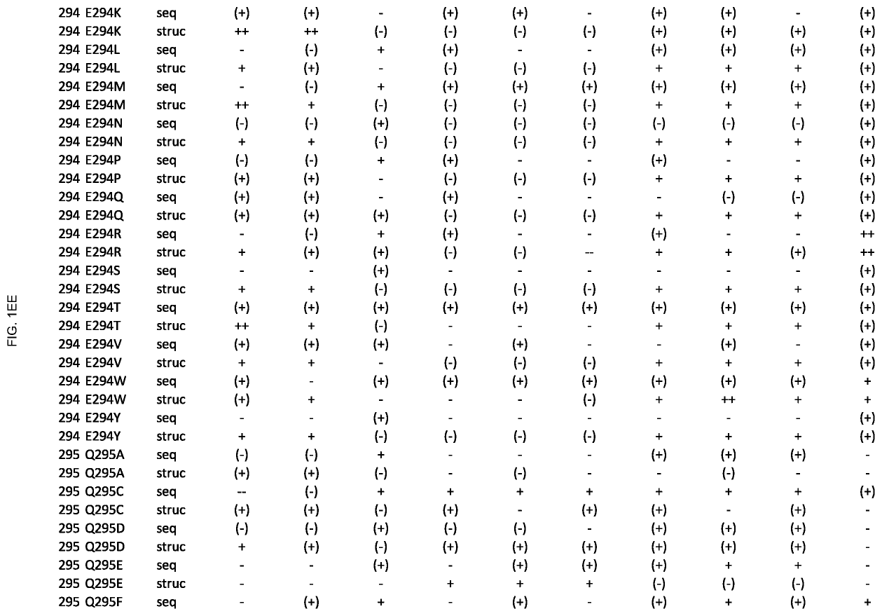

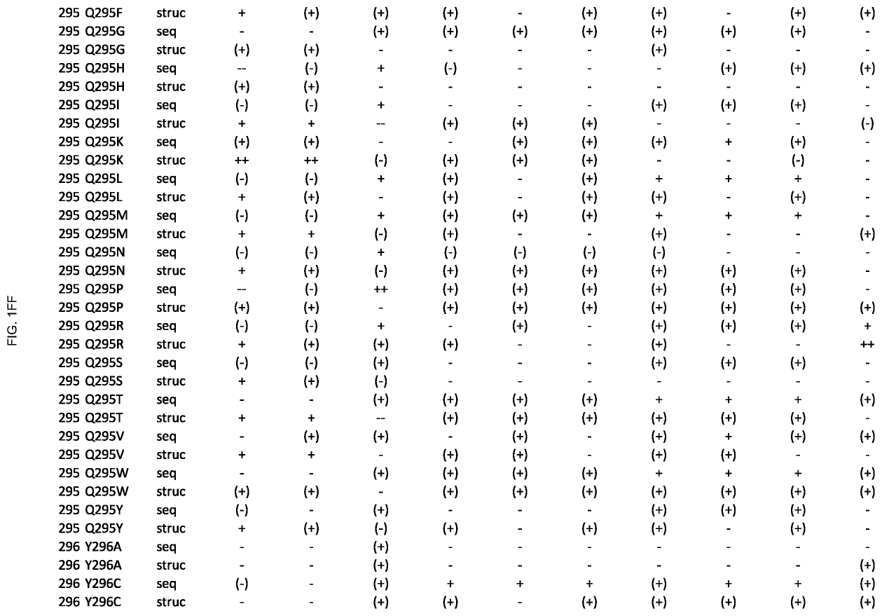

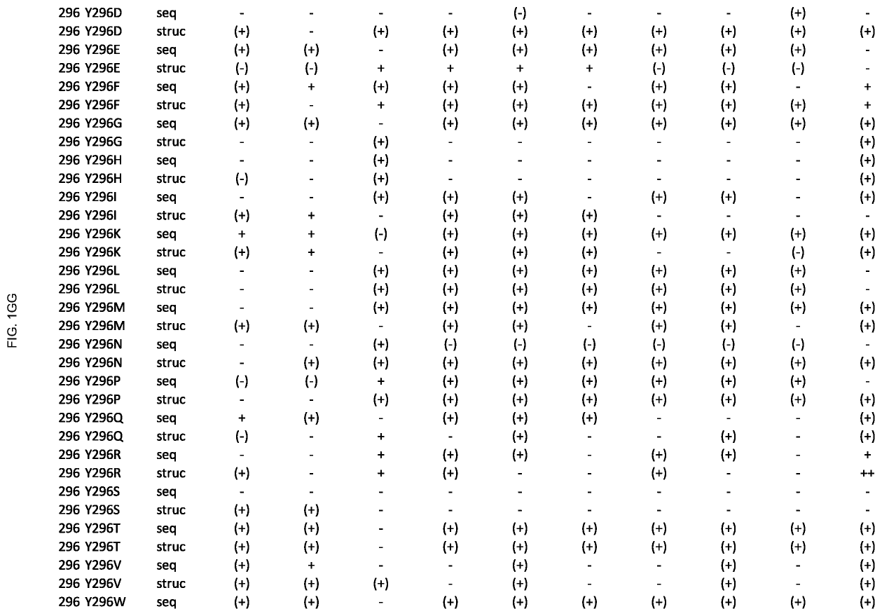

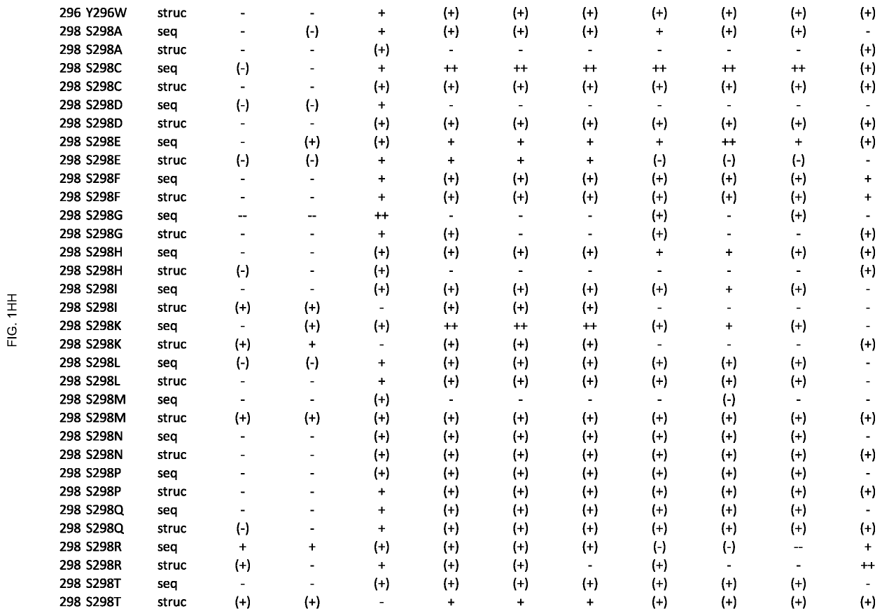

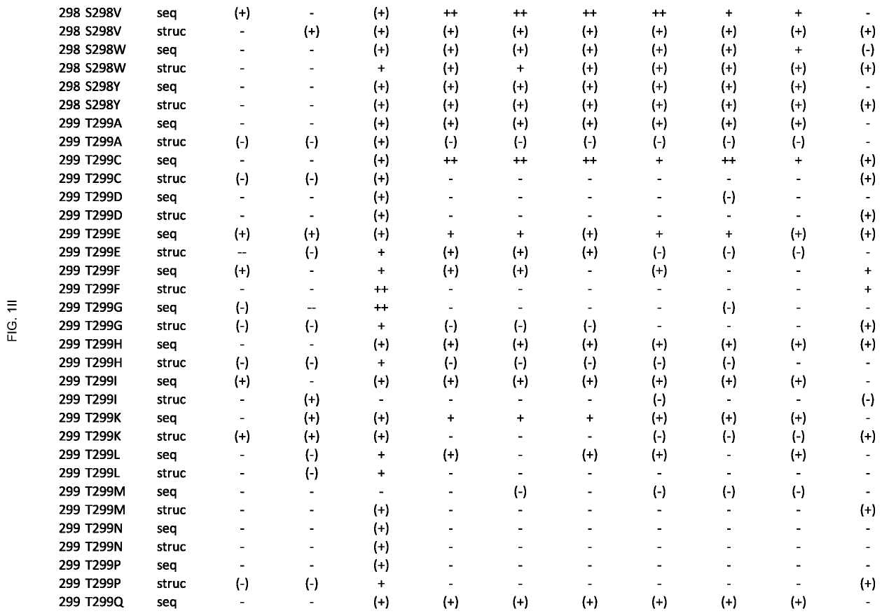

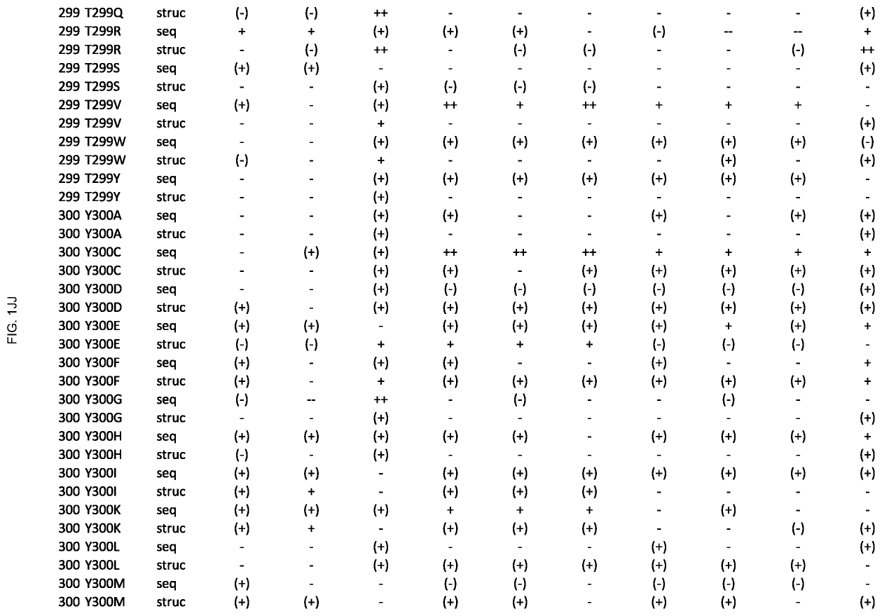

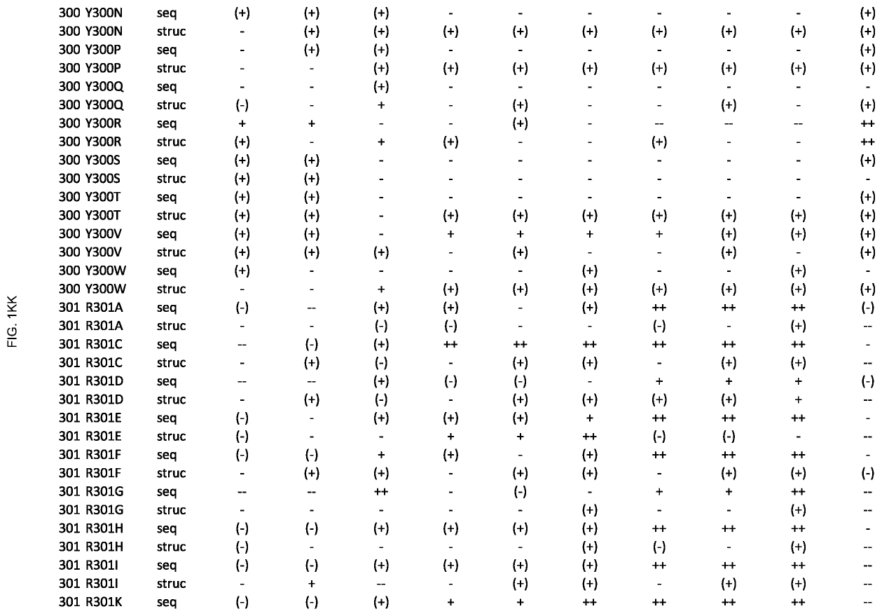

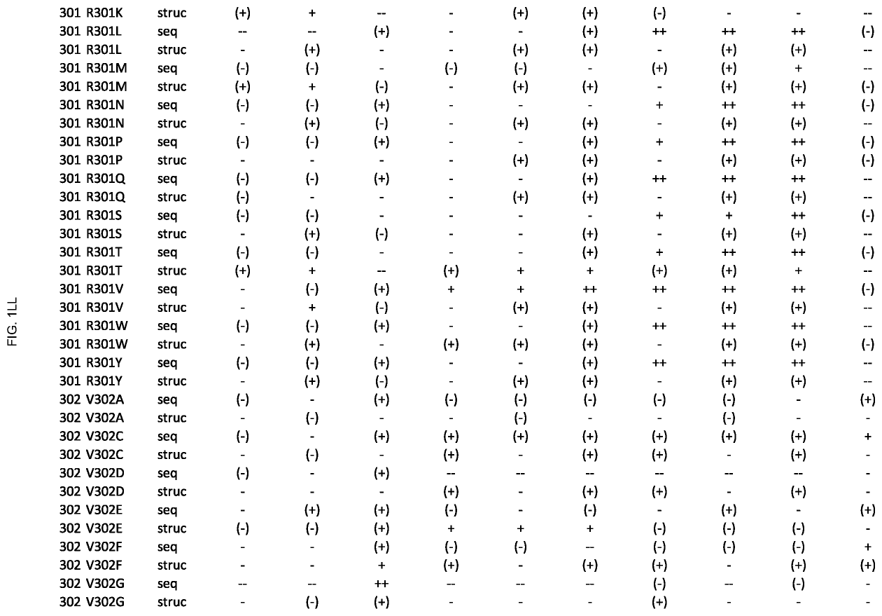

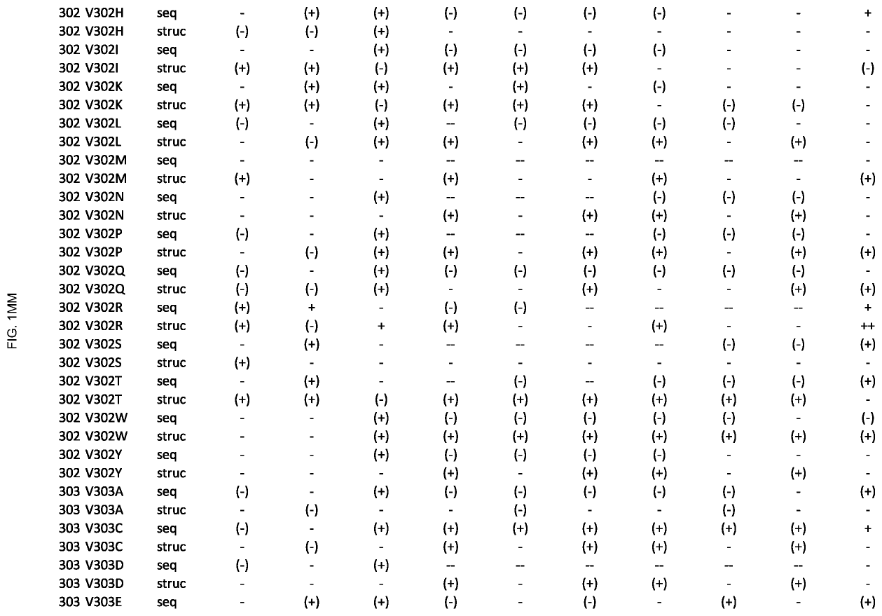

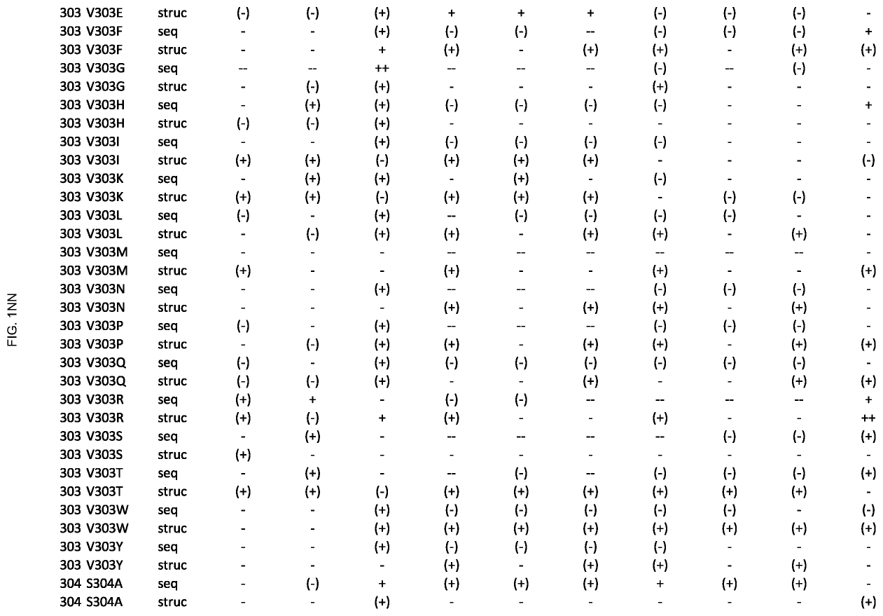

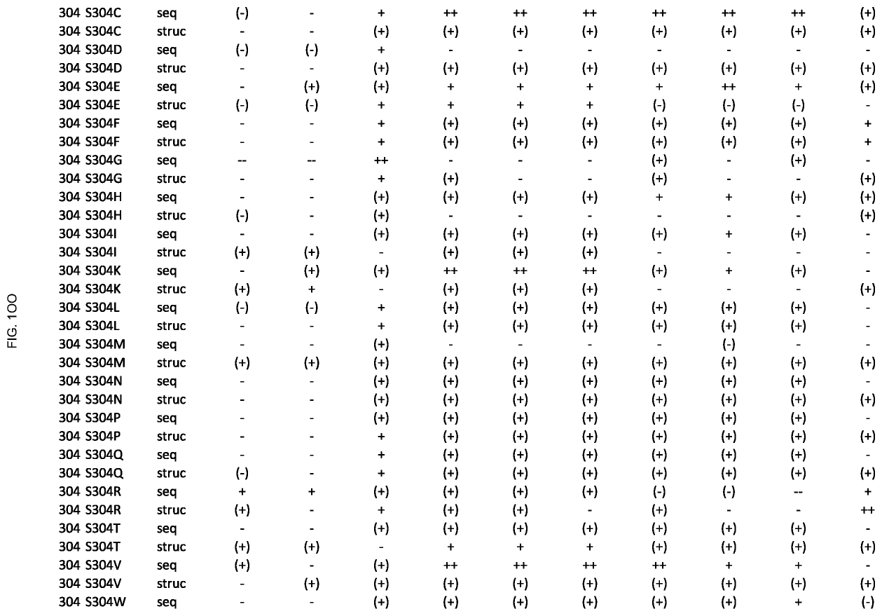

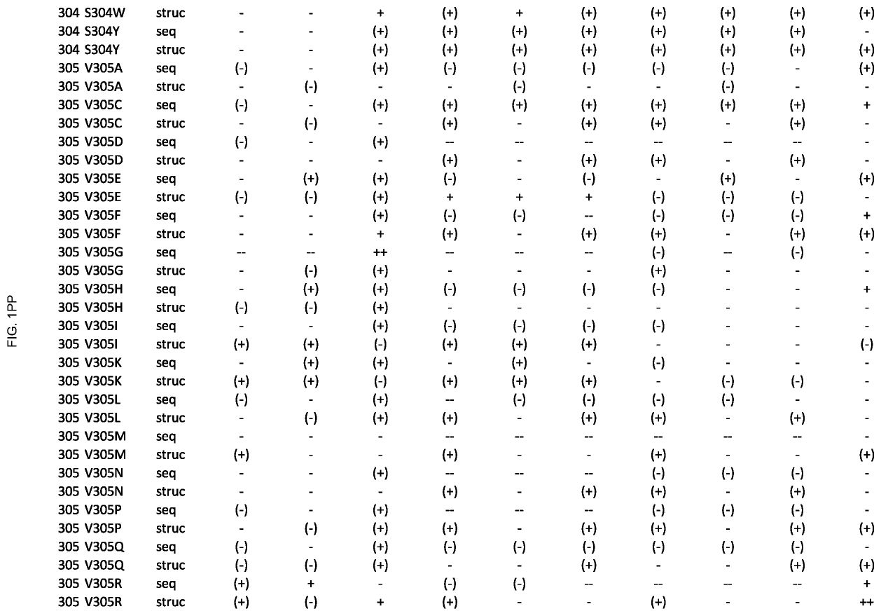

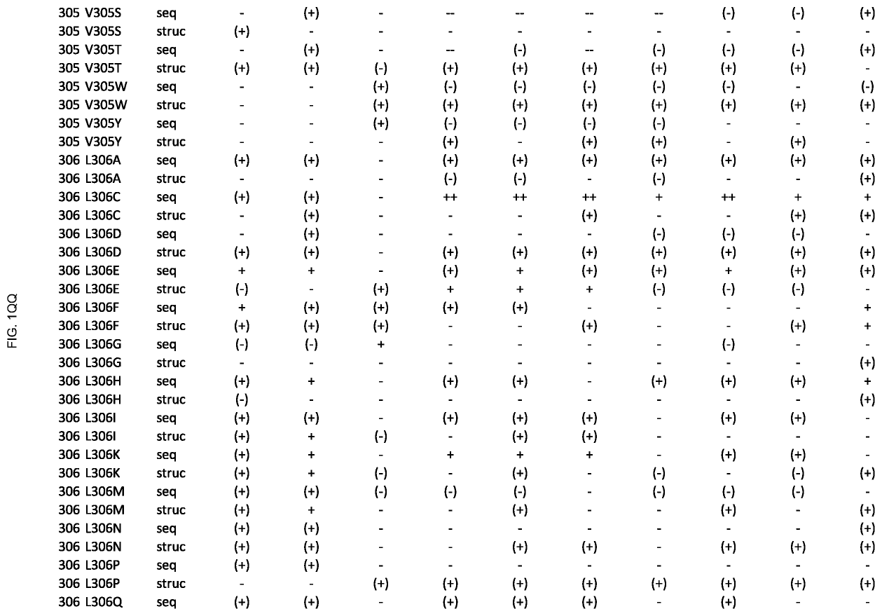

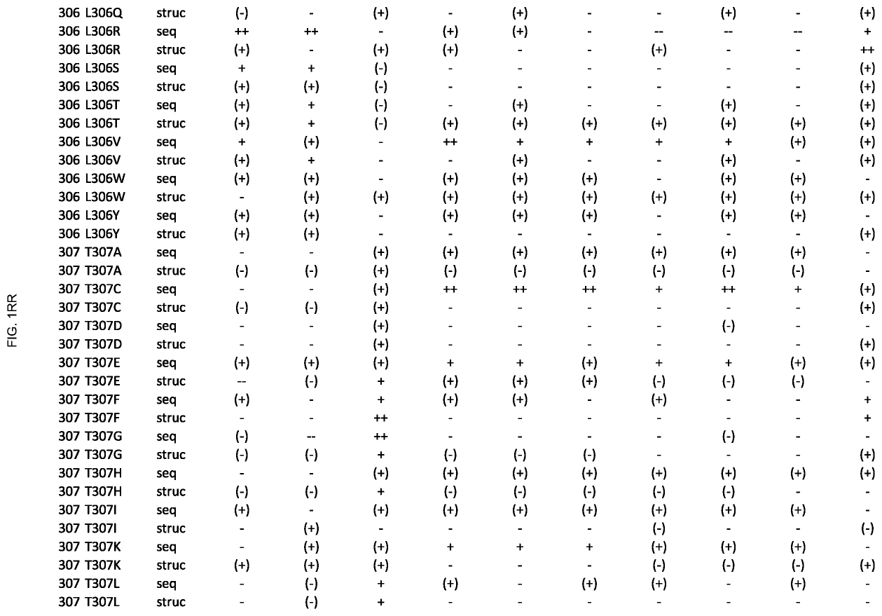

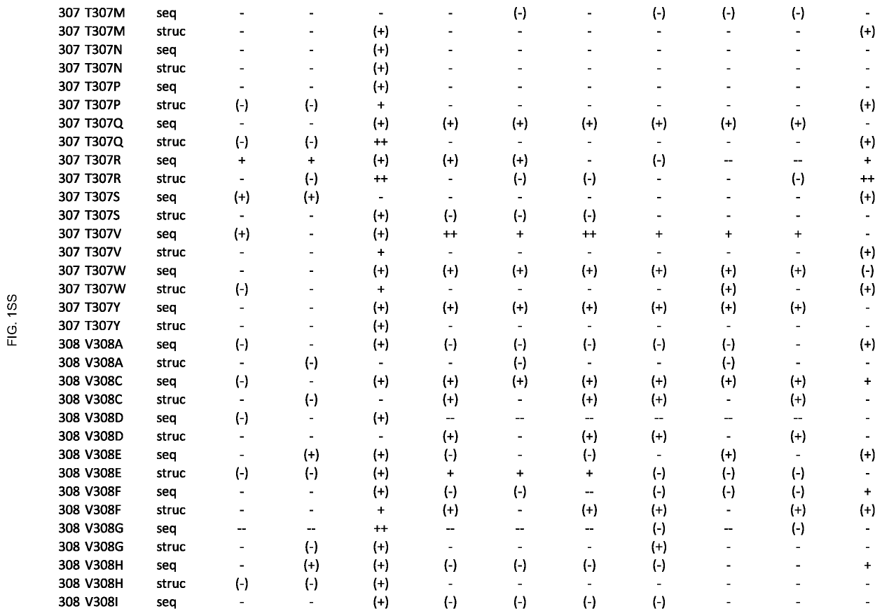

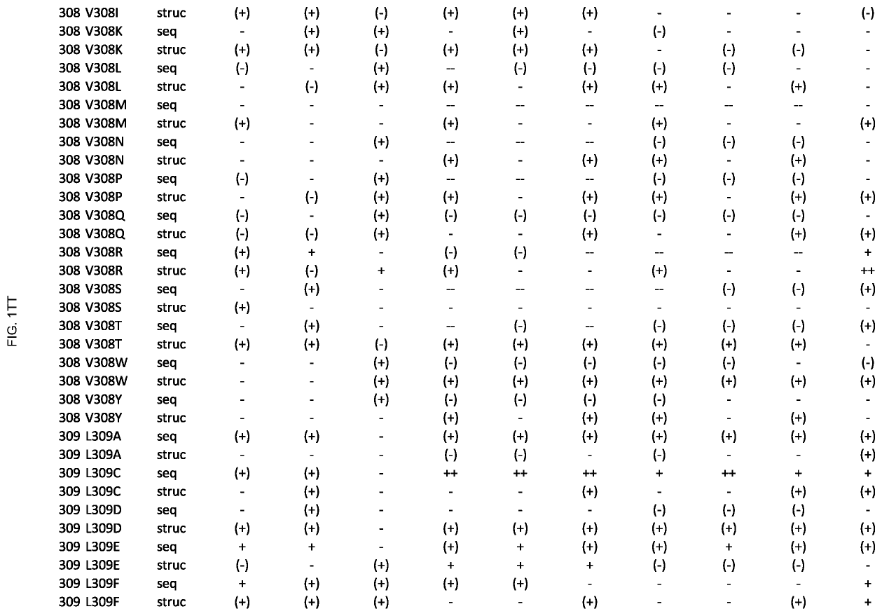

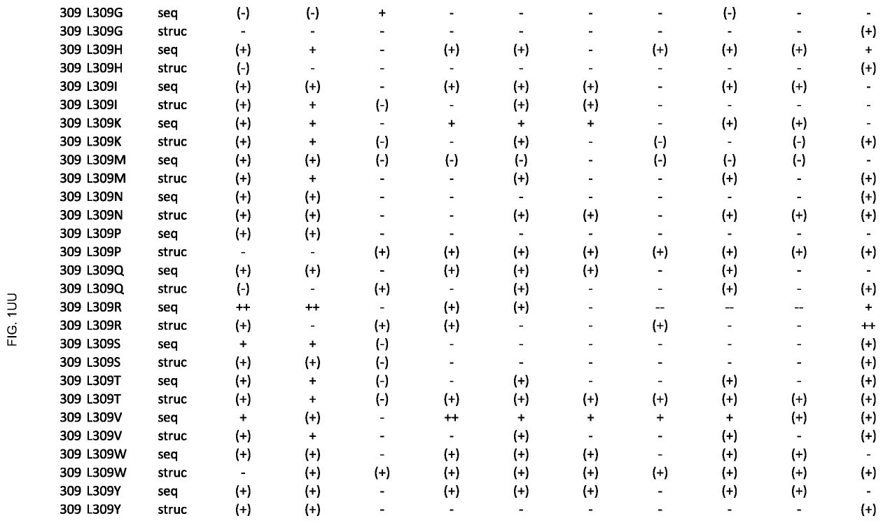

Additional experiments are performed where antibody Fc regions are mutated to alter glycosylation features of the Fc region. For instance, to change fucosylation (e.g., core-fucosylation), sialylation, bisection, branching, galactosylation, or oligomannose, or combinations thereof. Non-limiting example mutations are shown in Table 8 in FIGS. 1A-1UU. Table 8 shows substitutions possible for glycosite-proximal amino acids, and the expected change in terms of relative preference for competing glycan features. Seq refers to sequence, struc refers to structure. In some cases, the mechanism of modulating glycosylation feature is via sequence (seq) or structure (struc). ++ strong selection, + moderate selection, (+) weak selection, −− strong anti-selection, − moderate anti-selection, (−) weak anti-selection, where “selection” indicates that the substitution described in the row is consistent with the column header “glycan feature x is ‘preferred over’ or ‘selected over’ glycan feature y.” And anti-selection indicates the substitution is consistent with the opposite of the header “glycan feature y is preferred over glycan feature x”.

### Example 2: Modified Fc Regions have Altered Glycosylation

Rituximab antibodies having a substitution selected from: Q295E, Q295L, Y296R, S298K, or R301F, and wildtype (wt) Rituximab were expressed in ExpiCHO-S cells via transient transfection, purified using Mab-select columns, and measured antibody glycosylation using liquid chromatography (LC) and mass spectrometry (MS); glycans are released with PNGaseF, labeled with a fluorescent dye and analyzed by LC-MS. The mass signal was used to confirm the identity of the specific glycan and the peak area in the LC chromatogram as a measure of its relative abundance. Tables 9-14 provide the peak data for glycoprofiling of wildtype Rituximab as compared with Rituximab having a Q295E, Q295L, R301F, S298K, or Y296R substitution.

The glycan profiling method utilized measures glycan composition of the protein, but does not detect differences in glycan linkage. Therefore, differences between the glycan profile of a Rituximab variant as compared to wt Rituximab identified using this method are not indicative of differences in glycan linkages between the Rituximab variant(s) and wt Rituximab. While Rituximab variants Q295E and Q295L show minimal changes in glycan composition, changes in glycan linkage were not measured due to limitations of this method.

The Rituximab Y296R variant had more branching (A3F, A4F), more A1F relative to A2F, more high mannose species, and more non-fucosylation structures (A1 and A2), as compared to wt Rituximab.

The Rituximab S298K variant had more high mannose species, more A1F relative to A2F, and more non-fucosylation structures (A1 and A2), as compared to wt Rituximab.

The Rituximab R301F variant had more branching (A3F, A4F), more high mannose species, possibly more A2FG1, a decrease in A2F, and a small increase in A1, as compared to wt Rituximab.

The Rituximab Q295E variant showed a complete loss of A4F and a possible gain of A3F or A2FG1.

In the Rituximab Y296R variant, biantennary fucosylation decreases (more A2 than A2F) relative to wt Rituximab. In the Rituximab S298K variant, biantennary and monoantennary fucosylation both decrease (A2>A2F and A1>A1F). In Rituximab R301F, monoantennary fucosylation decreases (A1>A1F).

Overall, Q295E variants show a small decrease in afucosylation, Q295L shows a small possible decrease in terminal galactose. The Y296R and S298K variants show an increase in afucosylation, a small possible increase in terminal galactose and a decrease in terminal GlcNAc. The R301F variant shows an increase in afucosylation, an increase in possible terminal galactose, and an increase in terminal GlcNAc.

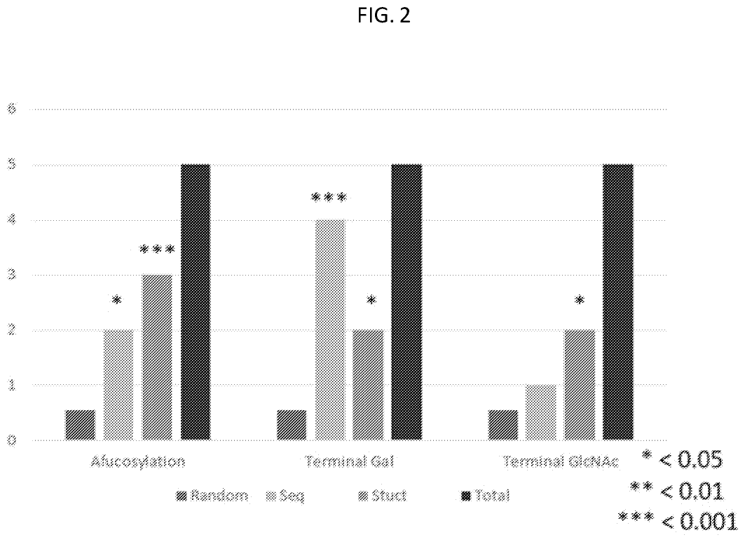

Using a binomial distribution, expected changes (outcomes: increase, decrease or neutral) in glycosylation resulting from particular substitutions in the Fc region of an antibody (as described in Table 8 in FIGS. 1A-1UU) were compared with (i) random changes in Fc glycosylation (increase, decrease or neutral) and (ii) experimentally observed changes (increase, decrease or neutral) in Fc glycosylation resulting from particular substitutions in the Fc region of the antibody (as described in this example). As shown in FIG. 2, expected changes in glycosylation of Fc regions with the particular substitutions (Q295E, Q295L, Y296R, S298K, or R301F) are 4- to 8-fold more consistent than random with observed changes in glycosylation in the Fc region. Sequence determined changes in fucosylation were 4-fold more consistent than random with observed changes (p<0.05), while structure determined changes were 6-fold more consistent than random with observed changes (p<0.001). Sequence determined changes in terminal galactose were 8-fold (p<0.001) more consistent than random with observed changes, and structure determined changes were 4-fold (p<0.05) more consistent than random with observed changes. Structure determined changes in terminal GlcNac were 4-fold (p<0.05) more consistent than random with observed changes.

TABLE 9
Peak list for Wildtype Rituximab

| N-Glycan | Apex RT | Start RT | End RT | Area | % Area | Height | % Height |
|---|---|---|---|---|---|---|---|
| A1 | 12.04 | 11.65 | 12.29 | 11534794.4 | 0.89 | 844691.5 | 0.94 |
| A1F | 13.23 | 12.82 | 13.49 | 58314042.2 | 4.47 | 4288457 | 4.78 |
| A2 | 13.89 | 13.53 | 14.1 | 15862112 | 1.22 | 1193421 | 1.33 |
| A2F | 14.95 | 14.38 | 15.35 | 891994292 | 68.45 | 63099040 | 70.38 |
| | 15.68 | 15.35 | 15.84 | 14212541.5 | 1.09 | 723525.3 | 0.81 |
| M5 | 16.3 | 15.84 | 16.65 | 109034125 | 8.37 | 6464686 | 7.21 |
| A3F/A2FG1 | 17.25 | 16.73 | 17.45 | 74500234 | 5.72 | 4262440 | 4.75 |
| | 17.56 | 17.45 | 17.74 | 18985297.5 | 1.46 | 1486328 | 1.66 |
| | 17.96 | 17.74 | 18.16 | 17732844.8 | 1.36 | 1033871 | 1.15 |
| A4F | 18.48 | 18.16 | 18.74 | 35357377.6 | 2.71 | 2126141 | 2.37 |

TABLE 10
Peak list for Rituximab with Q295E substitution

| N-Glycan | Apex RT | Start RT | End RT | Area | % Area | Height | % Height |
|---|---|---|---|---|---|---|---|
| A1 | 11.85 | 11.61 | 12.04 | 1582336 | 0.57 | 131288.1 | 0.65 |
| A1F | 12.98 | 12.74 | 13.21 | 6162679 | 2.24 | 466986.7 | 2.31 |
| A2 | 13.67 | 13.43 | 14.06 | 1267392 | 0.46 | 60793.2 | 0.3 |
| A2F | 14.69 | 14.28 | 15.11 | 2.09E+08 | 75.83 | 15639805 | 77.2 |
| M5 | 16 | 15.59 | 16.26 | 18509834 | 6.72 | 1223627 | 6.04 |
| A3F/A2FG1 | 16.96 | 16.6 | 17.09 | 15848821 | 5.76 | 1029835 | 5.08 |
| | 17.24 | 17.09 | 17.45 | 14995845 | 5.45 | 1117367 | 5.52 |
| | 17.67 | 17.46 | 17.91 | 4466606 | 1.62 | 285847 | 1.41 |
| A4F | 18.13 | 17.99 | 18.39 | 694279.9 | 0.25 | 54229.56 | 0.27 |

TABLE 11
Peak list for Rituximab with Q295L substitution

| N-Glycan | Apex RT | Start RT | End RT | Area | % Area | Height | % Height |
|---|---|---|---|---|---|---|---|
| A1 | 12.09 | 11.78 | 12.3 | 6078628 | 0.61 | 424261 | 0.68 |
| A1F | 13.27 | 12.92 | 13.56 | 31047752 | 3.14 | 2114478 | 3.39 |
| A2 | 13.93 | 13.58 | 14.15 | 6473850 | 0.65 | 445100.3 | 0.71 |
| A2F | 15 | 14.5 | 15.42 | 6.9E+08 | 69.72 | 44943268 | 72.07 |
| | 15.71 | 15.42 | 15.91 | 9987403 | 1.01 | 458658.7 | 0.74 |
| M5 | 16.34 | 15.91 | 16.7 | 85841778 | 8.68 | 4791097 | 7.68 |
| A3F/A2FG1 | 17.31 | 16.8 | 17.52 | 76476588 | 7.73 | 4011818 | 6.43 |
| | 17.6 | 17.52 | 17.84 | 27410983 | 2.77 | 2014700 | 3.23 |
| | 18.04 | 17.84 | 18.25 | 19216149 | 1.94 | 1111875 | 1.78 |
| A4F | 18.54 | 18.25 | 18.84 | 29322734 | 2.96 | 1671678 | 2.68 |
| | 22.59 | 22.34 | 22.94 | 7722454 | 0.78 | 374331.5 | 0.6 |

TABLE 12
Peak list for Rituximab with R301F substitution

| N-Glycan | Apex RT | Start RT | End RT | Area | % Area | Height | % Height |
|---|---|---|---|---|---|---|---|
| A1 | 12.06 | 11.76 | 12.27 | 8763346 | 0.91 | 673245.6 | 1.08 |
| A1F | 13.22 | 12.91 | 13.51 | 35857883 | 3.73 | 2473092 | 3.97 |
| A2 | 13.9 | 13.55 | 14.13 | 10992206 | 1.14 | 786543.1 | 1.26 |
| A2F | 14.97 | 14.38 | 15.35 | 4.73E+08 | 49.15 | 32311202 | 51.89 |
| | 15.69 | 15.35 | 15.88 | 18771071 | 1.95 | 1056367 | 1.7 |
| M5 | 16.31 | 15.88 | 16.67 | 1.05E+08 | 10.95 | 6082149 | 9.77 |
| A3F/A2FG1 | 17.27 | 16.72 | 17.46 | 1.48E+08 | 15.4 | 8447231 | 13.56 |
| | 17.56 | 17.48 | 17.78 | 38348035 | 3.99 | 3125887 | 5.02 |
| | 18 | 17.81 | 18.21 | 23018900 | 2.39 | 1388283 | 2.23 |
| A4F | 18.49 | 18.22 | 18.77 | 53075436 | 5.52 | 3243780 | 5.21 |
| | 19.86 | 19.36 | 20.13 | 31838320 | 3.31 | 1728465 | 2.78 |
| | 20.61 | 20.31 | 20.85 | 7205178 | 0.75 | 518131.8 | 0.83 |
| | 22.53 | 22.42 | 22.96 | 7764924 | 0.81 | 439020.9 | 0.7 |

TABLE 13
Peak list for Rituximab with S298K substitution

| N-Glycan | Apex RT | Start RT | End RT | Area | % Area | Height | % Height |
|---|---|---|---|---|---|---|---|
| A1 | 12.07 | 11.7 | 12.34 | 23562951 | 2.48 | 1842485 | 2.87 |
| A1F | 13.26 | 12.91 | 13.53 | 96843281 | 10.2 | 7549873 | 11.75 |
| A2 | 13.91 | 13.56 | 14.18 | 23856496 | 2.51 | 1799659 | 2.8 |
| A2F | 14.98 | 14.42 | 15.37 | 3.98E+08 | 41.89 | 28866064 | 44.94 |
| | 15.71 | 15.37 | 15.83 | 9350235 | 0.99 | 555784.8 | 0.87 |
| M5 | 16.33 | 15.83 | 16.75 | 1.89E+08 | 19.91 | 11574645 | 18.02 |
| A3F/A2FG1 | 17.28 | 16.79 | 17.48 | 45446890 | 4.79 | 2773198 | 4.32 |
| | 17.59 | 17.48 | 17.77 | 8365088 | 0.88 | 655571 | 1.02 |
| | 17.99 | 17.77 | 18.19 | 10625329 | 1.12 | 657756.7 | 1.02 |
| A4F | 18.52 | 18.19 | 18.76 | 22375629 | 2.36 | 1427731 | 2.22 |
| | 19.22 | 18.76 | 19.34 | 16992231 | 1.79 | 977657.6 | 1.52 |
| | 19.49 | 19.34 | 19.72 | 18209774 | 1.92 | 1259549 | 1.96 |
| | 22.16 | 21.83 | 22.21 | 7819714 | 0.82 | 516831.5 | 0.8 |
| | 22.57 | 22.21 | 22.91 | 24262405 | 2.56 | 1129737 | 1.76 |
| | 25.07 | 24.37 | 25.28 | 23764515 | 2.5 | 972857.1 | 1.51 |
| | 25.43 | 25.28 | 25.69 | 9134701 | 0.96 | 645119.7 | 1 |
| | 27.65 | 27.02 | 27.95 | 21847806 | 2.3 | 1027024 | 1.6 |

TABLE 14
Peak list for Rituximab with Y296R substitution

| N-Glycan | Apex RT | Start RT | End RT | Area | % Area | Height | % Height |
|---|---|---|---|---|---|---|---|
| A1 | 12.08 | 11.74 | 12.31 | 12187462 | 1.7 | 1009445 | 1.94 |
| A1F | 13.27 | 12.87 | 13.55 | 67062554.6 | 9.33 | 5380402 | 10.32 |
| A2 | 13.93 | 13.55 | 14.13 | 13772128.4 | 1.92 | 1123775 | 2.16 |
| A2F | 14.99 | 14.45 | 15.38 | 321027863 | 44.67 | 25056175 | 48.06 |
| | 15.72 | 15.38 | 15.86 | 11552015.8 | 1.61 | 730775.6 | 1.4 |
| M5 | 16.33 | 15.86 | 16.75 | 130126990 | 18.11 | 8549830 | 16.4 |
| A3F/A2FG1 | 17.29 | 16.77 | 17.51 | 54067744 | 7.52 | 3577637 | 6.86 |
| | 17.62 | 17.52 | 17.74 | 2344627.32 | 0.33 | 204458.4 | 0.39 |
| | 17.98 | 17.76 | 18.16 | 6741585.29 | 0.94 | 427665.1 | 0.82 |
| A4F | 18.53 | 18.18 | 18.75 | 31381451.6 | 4.37 | 2161197 | 4.15 |
| | 19.21 | 18.75 | 19.36 | 12653025.1 | 1.76 | 753531.9 | 1.45 |
| | 19.5 | 19.36 | 19.7 | 9092806.3 | 1.27 | 704249 | 1.35 |
| | 22.59 | 22.31 | 22.93 | 14259979.3 | 1.98 | 785503.7 | 1.51 |
| | 25.08 | 24.41 | 25.27 | 15010187.6 | 2.09 | 651625.2 | 1.25 |
| | 25.45 | 25.27 | 25.73 | 5821993.8 | 0.81 | 415547.5 | 0.8 |
| | 27.65 | 27.03 | 27.9 | 11614578.8 | 1.62 | 605696.7 | 1.16 |

### Example 3: Modified Fc Regions have Altered FcR Binding and Properties

Altered FcR binding: Rituximab antibodies having a substitution selected from: Q295E, Y296R, S298K, or Y296R, and wildtype (wt) Rituximab were tested for binding to Fc receptor FcγR1, FcγRIIA, and FcγRIIIA. Wildtype Rituximab has an Fc region of SEQ ID NO: 1.

Rituximab titers were determined in triplicate on an Octet® Red96 biolayer interferometry (BLI) instrument. Binding to ProA biosensors was recorded for 120 s at 30° C. Binding rates were converted to concentrations based on a standard curve generated using wildtype Rituximab, produced and purified in-house.

The Rituximab variants were purified by affinity chromatography using a 1-mL MAb Select Sure column (Cytiva) mounted on an Äkta Pure instrument. Equilibration and washing steps were performed using 20 mM sodium phosphate, 0.15 M NaCl, pH 7.2. The antibody was eluted with 0.1M Sodium citrate, pH 3. Elution fractions were neutralized with 0.2 V of 1 M Tris, pH 9. Next, the protein solutions were desalted using 5-mL Zeba™ Spin desalting columns (7K MWCO, Thermo Fisher) and dPBS as eluent. Finally, the desalted solutions were concentrated on 4-mL Amicon centrifugal filter units (50K MWCO, Millipore) aiming for a concentration of approximately 0.5 mg/mL. The final concentrations were determined by measuring absorbance at 280 nm on a Nanodrop 2000 spectrophotometer using an extinction coefficient of 1.46 (mg/mL)−1 cm−1.

The amount of protein recovered from the MAb Select affinity step was calculated from the area under the peak (UV trace, 280 nm) using the Unicorn evaluation software tool (Cytiva).

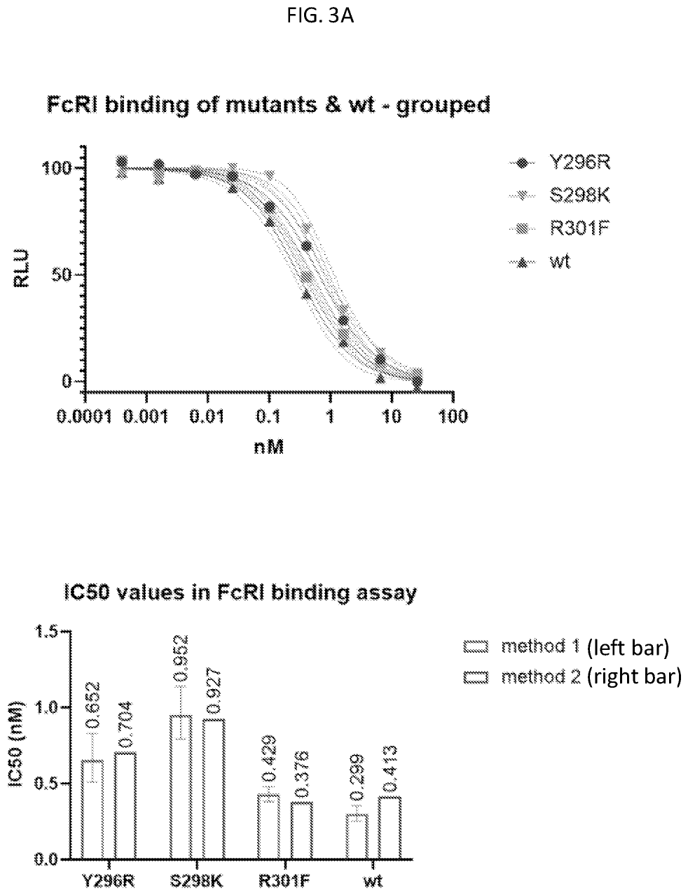

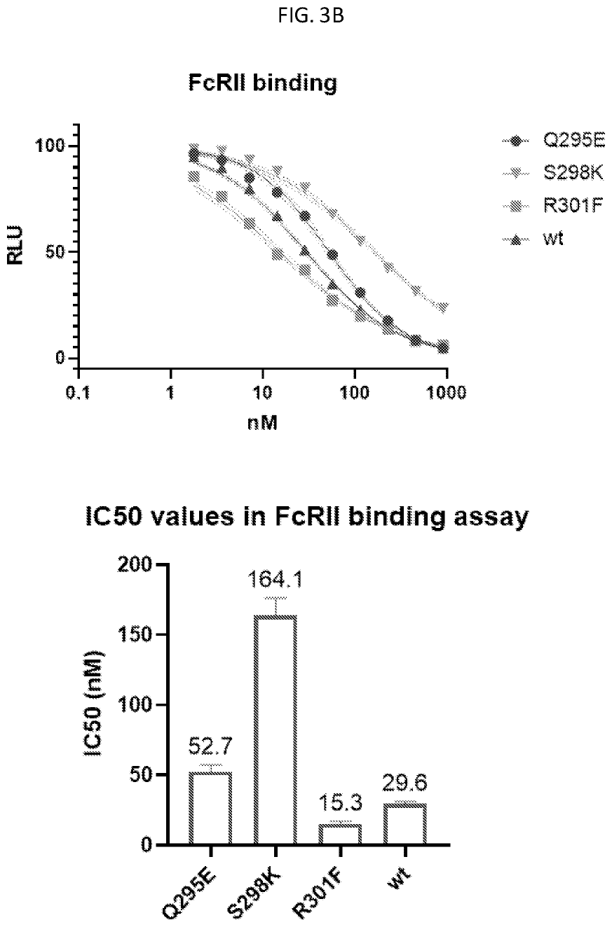

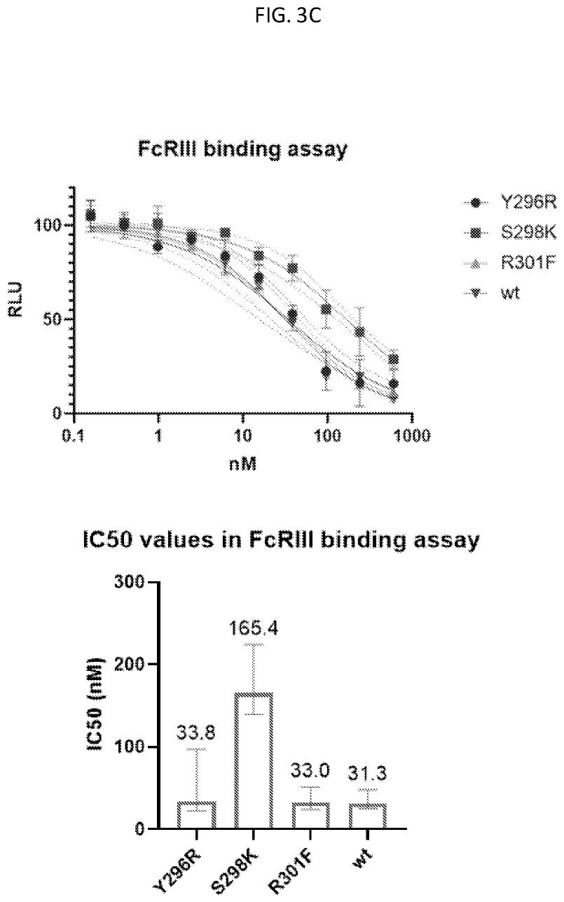

Binding to Fcγ receptor FcγRIIa (H131) was determined using the Lumit™ Fcγ receptor binding immunoassay from Promega (immunoassay CS3041A02). The assay was performed in white Nunc™ 96-well polypropylene microwell plates (Thermo Scientific, Cat #267350). Luminescence was measured on a BioTek Synergy Mx plate reader. Seven to ten reads per well were averaged and background subtracted, as determined from wells containing assay buffer with detection reagent only. Normalized luminescence was calculated by assigning 100% to the maximum luminescent signal obtained in the absence of analyte and then calculating percentage drop in signal in the presence of analyte. Data were fitted in GraphPad Prism (version 9.5.1) using the “[inhibitor] vs normalized response with variable slope” model in order to calculate IC50 values. 95% confidence intervals were calculated with separate lower and upper limit. They are shown as dotted lines in the graphs depicting the inhibition curves and as error bars in the bar graphs. FIG. 3A shows the change in binding to FcRI when the Fc region of Rituximab is altered with a Y296R, S298K, or R301F substitution. FIG. 3B shows the change in binding to FcRII when the Fc region of Rituximab is altered with a Q295E, S298K, or R301F substitution. For instance, R301F results in increased binding to FcRII as compared to wildtype. FIG. 3C shows the change in binding to FcRIII when the Fc region of Rituximab is altered with a S298K substitution.

Altered expressibility: Rituximab antibodies having a substitution selected from: Q295E, Q295L, Y296R, S298K, or Y296R, were tested for percent recovery as compared to wildtype (wt) Rituximab. Rituximab titers were determined in triplicate on an Octet® Red96 biolayer interferometry (BLI) instrument. Binding to ProA biosensors was recorded for 120 s at 30° C. Binding rates were converted to concentrations based on a standard curve generated using wildtype Rituximab, produced and purified, e.g., as described above. Variant Q295E showed a nearly 2-fold increase in expressibility over wildtype. Table 15 shows antibody titer (ug/ml) for various Rituximab mutants compared with wildtype Rituximab.

TABLE 15
Antibody expression

| | Titer | Average titer |
|---|---|---|
| Mutant | (ug/mL) | (μg/mL) |
| E294Q | 18.7 | 18.80 |
| | 18.8 | |
| | 18.9 | |
| Q295E | 25.1 | 24.83 |
| | 24.2 | |
| | 25.2 | |
| Q295H | 24 | 24.57 |
| | 25 | |
| | 24.7 | |
| Q295K | 16.6 | 15.50 |
| | 15.1 | |
| | 14.8 | |
| Q295L | 11.8 | 11.80 |
| | 12 | |
| | 11.6 | |
| Q295M | 8.98 | 8.95 |
| | 8.92 | |
| | 8.94 | |
| Q295V | 10.5 | 10.33 |
| | 10.4 | |
| | 10.1 | |
| Q295W | 13.9 | 14.13 |
| | 14.1 | |
| | 14.4 | |
| Q295Y | 8.63 | 8.74 |
| | 8.79 | |
| | 8.79 | |
| Y296C | 7.8 | 7.86 |
| | 7.92 | |
| | 7.86 | |
| Y296F | 6.3 | 6.39 |
| | 6.43 | |
| | 6.43 | |
| Y296H | 14.7 | 15.03 |
| | 15 | |
| | 15.4 | |
| Y296K | 7.34 | 7.26 |
| | 7.3 | |
| | 7.13 | |
| Y296R | 6.29 | 6.35 |
| | 6.32 | |
| | 6.43 | |
| Y296W | 5.01 | 5.07 |
| | 5.16 | |
| | 5.03 | |
| S298F | 17.8 | 17.40 |
| | 17.3 | |
| | 17.1 | |
| S298I | 15.8 | 15.83 |
| | 16 | |
| | 15.7 | |
| S298K | 14.1 | 13.73 |
| | 13.7 | |
| | 13.4 | |
| S298Q | 18.7 | 17.87 |
| | 18.3 | |
| | 16.6 | |
| S298W | 10.7 | 10.37 |
| | 10.5 | |
| | 9.92 | |
| Y300F | 13.5 | 13.47 |
| | 13.6 | |
| | 13.3 | |
| Y300Q | 14.5 | 13.97 |
| | 14.2 | |
| | 13.2 | |
| Y300R | 20.9 | 20.83 |
| | 21.4 | |
| | 20.2 | |
| Y300W | 17.5 | 16.53 |
| | 16.2 | |
| | 15.9 | |
| R301D | 15.9 | 15.67 |
| | 15.8 | |
| | 15.3 | |
| R301F | 10.7 | 10.53 |
| | 10.6 | |
| | 10.3 | |
| R301G | 9.62 | 9.61 |
| | 9.63 | |
| | 9.58 | |
| R301H | 9.19 | 9.15 |
| | 9.21 | |
| | 9.04 | |
| R301Y | 7.21 | 7.27 |
| | 7.24 | |
| | 7.37 | |
| V302H | 10.4 | 10.27 |
| | 10.2 | |
| | 10.2 | |
| wildtype | 14.1 | 13.80 |
| | 13.7 | |
| | 13.6 | |

Percent recovery for purified Rituximab mutants as compared to wildtype Rituximab was measured, results are shown in Table 16. Percent recovery was measured using Octet.

TABLE 16
Antibody Recovery

| | Yield (mg) (30 ml | Predicted yield | % |
|---|---|---|---|
| Sample | sample) | based on Octet | Recovery |
| Q295E | 0.39 | 0.74 | 52 |
| Q295L | 0.23 | 0.35 | 65 |
| Y296R | 0.10 | 0.19 | 52 |
| S298K | 0.28 | 0.41 | 68 |
| R301F | 0.22 | 0.32 | 70 |
| WT | 0.24 | 0.41 | 58 |

### Example 3: Modified Fc Regions in Rituximab do not Interfere with Antigen Binding

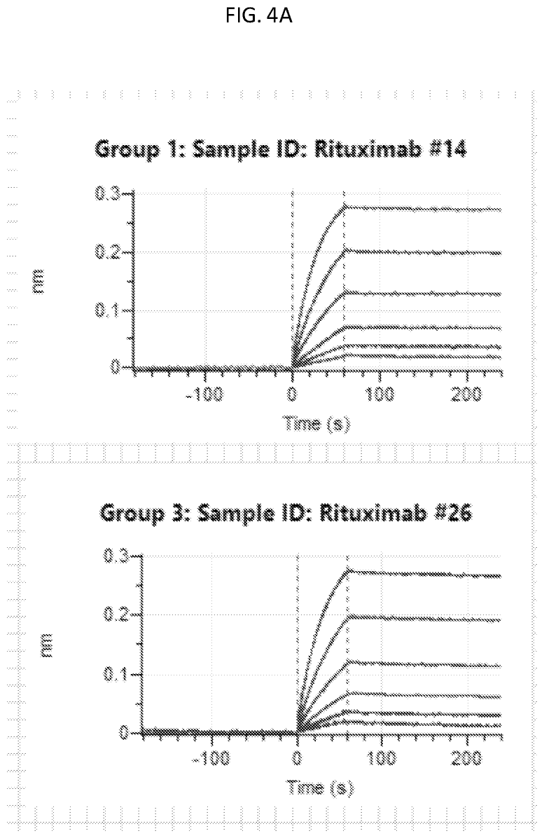

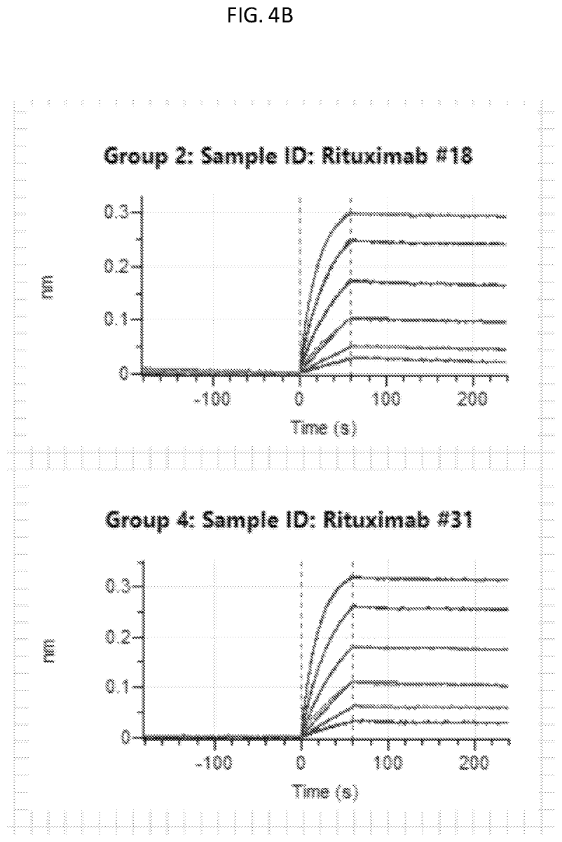

Binding of Rituximab antibodies having a Fc substitution (Y296R, S298K, or R301F), or wildtype (wt) Rituximab to CD20 antigen was tested using Octet RED96e instrument. The CD20 antigen tested is full-length, biotinylated, human CD20 (Acro Biosystems). The binding assay was performed per standard protocol by Acro Biosystems. Briefly, biosensors were pre-loaded with CD20 and dipped successively in buffer (baseline, 180 s), Rituximab or Rituximab variant (3.13, 6.25, 12.5, 25, 50 and 100 nM, 60 s), and again in buffer (dissociation step, 180 s). The sensograms are shown in FIGS. 4A-4B (Rituximab #14=Y296R, Rituximab #18=S298K, Rituximab #26=R301F, Rituximab #31=wildtype). All Rituximab mutants show binding to CD20, with Kd values similar to the Kd reported by Acro Biosystems (2.47E−10). Table 17 shows the results of the kinetic analysis of the three highest concentrations for each Rituximab variant.

TABLE 17
Rituximab antigen binding

| Sample ID | Conc. (nM) | KD (M) | KD Error |
|---|---|---|---|
| Rituximab #31 | 100 | 1.69E−10 | 6.27E−12 |
| Rituximab #31 | 50 | 1.69E−10 | 6.39E−12 |
| Rituximab #31 | 25 | 1.71E−10 | 8.23E−12 |
| Rituximab #14 | 100 | 2.55E−10 | 1.15E−11 |
| Rituximab #14 | 50 | 1.94E−10 | 1.46E−11 |
| Rituximab #14 | 25 | 1.31E−10 | 1.29E−11 |
| Rituximab #18 | 100 | 2.23E−10 | 7.22E−12 |
| Rituximab #18 | 50 | 2.41E−10 | 9.48E−12 |
| Rituximab #18 | 25 | 3.01E−10 | 9.54E−12 |
| Rituximab #26 | 100 | 4.78E−10 | 1.58E−11 |
| Rituximab #26 | 50 | 3.63E−10 | 1.58E−11 |
| Rituximab #26 | 25 | 4.55E−10 | 1.59E−11 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An antibody Fc region comprising a substitution(s): R301, Q295, Y296, S298, K290, P291, R292, E293, E294, Y300, V302, V303, S304, V303, L306, T307, V308, L309, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

2. An antibody Fc region comprising a substitution(s): Q295E, P291E, P291K, P291Q, R292E, R292K, R292Q, Y296E, Y296K, Y296Q, R301E, R301K, R301Q, P291I& V303F, V302F&V303F, P291Q&V303F, P291L&V303F, P291L&S304N, V303F&S304N, V303F&S304T, V303F&S304F, P291L&V303W, P291Q&S304N, P291I&V303W, V302W&V303F, V302W&V303W, V302F&V303W, P291I&V302F, P291Q&S304F, P291Q&R292Q, P291L&S304F, P291Q&S304T, P291L&V303Q, V302H&V303F, R292Q&V303F, P291Q&V302F, P291L&V302F, V302Q&V303F, P291I&V303Q, V302Y&V303W, P291I &S304N, V302F&S304N, V302Q&V303W, K290L&P291L, P291Q&T207N, K290L&V303Q, K290L&V303W, K290I&P291L, R292Q&T207N, P291E&T207N, K290L&V303F, K290L&V303Y, K290L&V303I, K290I&V303F, K290I&V303Q, P291L&T207N, K290L&P291Q, K290E&V303F, K290E&R292Q, K290L&V302Q, K290L&R292Q, K290L&V303H, K290L&V302W, K290E&V303Q, K290E&V303Y, K290L&V302I, K290L&V302Y, K290E&V303H, K290E&V302Q, K290L&V302F, K290I&V302Q, K290I&V303W, K290E&P291L, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution decreases core-fucosylation at amino acid N297 as compared to the antibody Fc region without the substitution.

3. An antibody Fc region comprising a substitution at position(s): Q295K, Q295R, Y296F, and/or Y300F, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution increases core-fucosylation at amino acid N297 as compared to the antibody Fc region without the substitution.

4. An antibody Fc region comprising a substitution at position(s): Y296R, P291R, and/or Y296W, according to the EU numbering system; optionally wherein: the substitution does not alter an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution does not alter core-fucosylation at amino acid N297 as compared to the antibody Fc region without the substitution.

5. An antibody Fc region comprising a substitution(s): P291I, P291L, P291V, Y296I, Y296V, S298F, S298H, S298N, S298T, S298W, S298Y, Y300F, Y300H, Y300I, Y300I, Y300L, Y300V, Y300V, Y300W, Y300W, R301H, R301W, V302F, V302H, V302I, V302L, V302Q, V302W, V302Y, V303F, V303H, V303I, V303L, V303Q, V303W, V303Y, S304F, S304H, S304N, S304T, S304W, S304Y, V305H, V305K, V305Q, V305R, or V305W, or any combination thereof, wherein the numbering is according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution alters core-fucosylation at amino acid N297 as compared to the antibody Fc region without the substitution.

6. An antibody Fc region comprising a substitution(s): Q295E, P291E, P291K, P291Q, R292E, R292K, R292Q, Y296E, Y296K, Y296Q, R301E, R301K, R301Q, P291I& V303F, V302F&V303F, P291Q&V303F, P291L&V303F, P291L&S304N, V303F&S304N, V303F&S304T, V303F&S304F, P291L&V303W, P291Q&S304N, P291I&V303W, V302W&V303F, V302W&V303W, V302F&V303W, P291I&V302F, P291Q&S304F, P291Q&R292Q, P291L&S304F, P291Q&S304T, P291L&V303Q, V302H&V303F, R292Q&V303F, P291Q&V302F, P291L&V302F, V302Q&V303F, P291I&V303Q, V302Y&V303W, P291I&S304N, V302F&S304N, V302Q&V303W, K290L&P291L, P291Q&T207N, K290L&V303Q, K290L&V303W, K290I&P291L, R292Q&T207N, P291E&T207N, K290L&V303F, K290L&V303Y, K290L&V303I, K290I&V303F, K290I&V303Q, P291L&T207N, K290L&P291Q, K290E&V303F, K290E&R292Q, K290L&V302Q, K290L&R292Q, K290L&V303H, K290L&V302W, K290E&V303Q, K290E&V303Y, K290L&V302I, K290L&V302Y, K290E&V303H, K290E&V302Q, K290L&V302F, K290I&V302Q, K290I&V303W, K290E&P291L, Q295K, Q295R, Y296F, Y300F, P291I, P291L, P291V, Y296I, Y296V, S298F, S298H, S298N, S298T, S298W, S298Y, Y300F, Y300H, Y300I, Y300I, Y300L, Y300V, Y300V, Y300W, Y300W, R301H, R301W, V302F, V302H, V302I, V302L, V302Q, V302W, V302Y, V303F, V303H, V303I, V303L, V303Q, V303W, V303Y, S304F, S304H, S304N, S304T, S304W, S304Y, V305H, V305K, V305Q, V305R, V305W, or any combination thereof, wherein the numbering is according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution alters core-fucosylation at amino acid N297 as compared to the antibody Fc region without the substitution.

7. An antibody Fc region comprising a substitution at one or more position(s) shown in a Table herein, wherein the numbering is according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution alters core-fucosylation at amino acid N297 as compared to the antibody Fc region without the substitution.

8. An antibody Fc region comprising a substitution(s): Q295E, P291E, P291K, P291Q, R292E, R292K, R292Q, Y296E, Y296K, Y296Q, R301E, R301K, R301Q, P291I, V303F, V302F, P291L, S304N, S304T, S304F, V303W, V302W, R292Q, V303Q, V302H, V302Q, V302Y, K290L, T207N, K290I, V303Y, V303I, K290E, V303H, V302I, Q295K, Q295R, Y296F, Y300F, P291V, Y296I, Y296V, S298F, S298H, S298N, S298T, S298W, S298Y, Y300H, Y300I, Y300L, Y300V, Y300W, R301H, R301W, V302L, V303L, S304H, S304W, S304Y, V305H, V305K, V305Q, V305R, V305W, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

9. An antibody Fc region comprising a substitution(s): Q295E, P291E, P291K, P291Q, R292E, R292K, R292Q, Y296E, Y296K, Y296Q, R301E, R301K, R301Q, P291I& V303F, V302F&V303F, P291Q&V303F, P291L&V303F, P291L&S304N, V303F&S304N, V303F&S304T, V303F&S304F, P291L&V303W, P291Q&S304N, P291I&V303W, V302W&V303F, V302W&V303W, V302F&V303W, P291I&V302F, P291Q&S304F, P291Q&R292Q, P291L&S304F, P291Q&S304T, P291L&V303Q, V302H&V303F, R292Q&V303F, P291Q&V302F, P291L&V302F, V302Q&V303F, P291I&V303Q, V302Y&V303W, P291I&S304N, V302F&S304N, V302Q&V303W, K290L&P291L, P291Q&T207N, K290L&V303Q, K290L&V303W, K290I&P291L, R292Q&T207N, P291E&T207N, K290L&V303F, K290L&V303Y, K290L&V303I, K290I&V303F, K290I&V303Q, P291L&T207N, K290L&P291Q, K290E&V303F, K290E&R292Q, K290L&V302Q, K290L&R292Q, K290L&V303H, K290L&V302W, K290E&V303Q, K290E&V303Y, K290L&V302I, K290L&V302Y, K290E&V303H, K290E&V302Q, K290L&V302F, K290I&V302Q, K290I&V303W, K290E&P291L, Q295K, Q295R, Y296F, Y300F, P291I, P291L, P291V, Y296I, Y296V, S298F, S298H, S298N, S298T, S298W, S298Y, Y300F, Y300H, Y300I, Y300I, Y300L, Y300V, Y300V, Y300W, Y300W, R301H, R301W, V302F, V302H, V302I, V302L, V302Q, V302W, V302Y, V303F, V303H, V303I, V303L, V303Q, V303W, V303Y, S304F, S304H, S304N, S304T, S304W, S304Y, V305H, V305K, V305Q, V305R, V305W, or any combination thereof, according to the EU numbering system; optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

10. The antibody Fc region of any one of claims 1-9, wherein the antibody Fc region without the substitution comprises SEQ ID NO: 1.

11. An antibody comprising the antibody Fc region of any one of claims 1-10, optionally where the antibody is IgG1, IgG2, IgG3, or IgG4.

12. A method of treating a disease or condition in a subject in need thereof, the method comprising administering to the subject a composition comprising the antibody Fc region of any one of claims 1-10, or the antibody of claim 11.

13. An antibody Fc region comprising a deletion or substitution within 10 amino acids of a N297 glycosite, wherein:
(a) the substitution(s) and/or deletion(s) are upstream of the glycosite comprising the removal of K, P, R, E, Q, or Y, or any combination thereof,
(b) the substitution(s) and/or deletion(s) are downstream of the glycosite comprising the removal of S, Y, R, V, L, K, or T, or any combination thereof,
(c) the substitution(s) and/or deletion(s) are upstream and/or downstream of the glycosite comprising the removal of K, P, R, E, Q, Y, S, V, L, or T, or any combination thereof,
(d) the substitution(s) and/or deletions(s) comprise removal of K 7 positions upstream of the glycosite, P 6 positions upstream of the glycosite, R 5 positions upstream of the glycosite, E 4 positions upstream of the glycosite, E 3 positions upstream of the glycosite, Q 2 positions upstream of the glycosite, Y 1 positions upstream of the glycosite, S 1 positions downstream of the glycosite, Y 3 positions downstream of the glycosite, R 4 positions downstream of the glycosite, V 5 positions downstream of the glycosite, V 6 positions downstream of the glycosite, S 7 positions downstream of the glycosite, V 8 positions downstream of the glycosite, L 9 positions downstream of the glycosite, T 10 positions downstream of the glycosite, V 11 positions downstream of the glycosite, or L 112 positions downstream of the glycosite, or any combination thereof,
(e) the substitution(s) and/or insertions(s) are upstream of the glycosite comprising the addition of E, K, Q, I, L, or V, or any combination thereof,
(f) the substitution(s) and/or deletion(s) are downstream of the glycosite comprising the removal of E, K, Q, P, L, F, T, N, W, H, or Y, or any combination thereof,
(g) the substitution(s) and/or deletion(s) are upstream and/or downstream of the glycosite comprising the removal of E, K, Q, P, I, V, L, F, T, N, W, H, or Y, or any combination thereof, and/or
(h) the substitution(s) and/or insertion(s) comprise addition of I, L, E, K, or Q 7 positions upstream of the glycosite, I, L, V, E, K, or Q 6 positions upstream of the glycosite, I, L, V, E, K, or Q 5 positions upstream of the glycosite, E, R or K 2 positions upstream of the glycosite, E, K, Q, F, I, L, V 1 position upstream of the glycosite, F, H, N, T, W, Y 1 position downstream of the glycosite, F, H, I, L, V, W 3 position downstream of the glycosite, E, K, Q, H, W 4 positions downstream of the glycosite, F, W, H, Q, Y, I, L 5 position downstream of the glycosite, F, W, H, Q, Y, I, L 6 position downstream of the glycosite, N, T, F, H, W, Y 6 position downstream of the glycosite, or N, H, K, Q, R, W 9 position downstream of the glycosite, or any combination thereof,
optionally wherein: the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution decreases core-fucosylation at amino acid N297 as compared to the antibody Fc region without the substitution, the substitution increases core-fucosylation at amino acid N297 as compared to the antibody Fc region without the substitution, and/or the substitution changes glycosylation features at amino acid N297 as compared to the antibody Fc region without the substitution.

14. An antibody Fc region comprising one or more substitutions in Table 8, according to the EU numbering system; optionally wherein: the substitution alters a glycosylation feature as compared to the antibody Fc region without the substitution, the substitution alters an effector function as compared to the antibody Fc region without the substitution, the substitution increases ADCC as compared to the antibody Fc region without the substitution, the substitution decreases ADCC as compared to the antibody Fc region without the substitution, the substitution increases CDC as compared to the antibody Fc region without the substitution, the substitution decreases CDC as compared to the antibody Fc region without the substitution, the substitution increases ADCP as compared to the antibody Fc region without the substitution, the substitution decreases ADCP as compared to the antibody Fc region without the substitution, the substitution changes glycosylation features at amino acid N297 as compared to the antibody Fc region without the substitution, and/or the substitution decreases core-fucosylation at amino acid N297 as compared to the antibody Fc region without the substitution.

15. The antibody Fc region of claim 13 or claim 14, wherein the antibody Fc region without the substitution comprises SEQ ID NO: 1.

16. An antibody comprising the antibody Fc region of any one of claims 13-15, optionally where the antibody is IgG1, IgG2, IgG3, or IgG4.

17. A method of treating a disease or condition in a subject in need thereof, the method comprising administering to the subject a composition comprising the antibody Fc region of any one of claims 13-15, or the antibody of claim 16.


SEQUENCE LISTING (2 sequences)

SEQ ID NO: 1 - protein, 330 aa, synthetic construct [sequence omitted]

SEQ ID NO: 2 - protein, 17 aa, synthetic construct
PREEQYNSTYRVVSVLT